SEQUENCE LISTING (37 sequences)

SEQ ID NO: 1 - protein, 15 aa, Artificial
  chemically synthesized
SGGGGSGGGGSGGGG

SEQ ID NO: 2 - protein, 25 aa, Artificial
  chemically synthesized
SGGGGSGGGGSGGGGSGGGGSGGGG

SEQ ID NO: 3 - protein, 16 aa, Artificial
  chemically synthesized
GQQQQGQQQQGQQQQG

SEQ ID NO: 4 - protein, 15 aa, Artificial
  chemically synthesized
GQQQQGQQQQGQQQQ

SEQ ID NO: 5 - protein, 17 aa, Artificial
  chemically synthesized
GSSSSSSSSSSSSSSSG

SEQ ID NO: 6 - protein, 3 aa, Artificial
  chemically synthesized
GST

SEQ ID NO: 7 - protein, 18 aa, Artificial
  chemically synthesized
GGGGSGGGGSGGGGSGAS

SEQ ID NO: 8 - protein, 19 aa, Artificial
  chemically synthesized
GSSSSSSSSSSSSSSSGAS

SEQ ID NO: 9 - protein, 16 aa, Artificial
  chemically synthesized
GGGGSGGGGSGGGGSG

SEQ ID NO: 10 - protein, 26 aa, Artificial
  chemically synthesized
GGGGSGGGGSGGGGSGGGGSGGGGSG

SEQ ID NO: 11 - protein, 17 aa, Artificial
  chemically synthesized
GGGGSGGGGSGGGGSGG

SEQ ID NO: 12 - protein, 27 aa, Artificial
  chemically synthesized
GGGGSGGGGSGGGGSGGGGSGGGGSGG

SEQ ID NO: 13 - DNA, 21 bp, Artificial
  chemically synthesized
agatcttttgccaccgctagc

SEQ ID NO: 14 - DNA, 30 bp, Artificial
  chemically synthesized
aagcttgtccccgggcaaatgagtgctagc

SEQ ID NO: 15 - DNA, 24 bp, Artificial
  chemically synthesized
agatctatatatatatatgctagc

SEQ ID NO: 16 - protein, 11 aa, Artificial
  chemically synthesized
RTVAAPSVFIF

SEQ ID NO: 17 - DNA, 38 bp, Artificial
  chemically synthesized
aagcttcaacaggggagagtgttgaagggagaggcgcc

SEQ ID NO: 18 - protein, 345 aa, Human immunodeficiency virus
  misc_feature: Locus HIVH3BH8; HIV-1 isolate LAI/IIIB clone BH8 from France; Ratner, L. et al., Nature 313 (1985) 277-384
AVGIGALFLGFLGAAGSTMGAASMTLTVQARQLLSGIVQQQNNLLRAIEGQQHLLQLTVWGIKQLQARILAVERYLKDQQLLGIWGCSGKLICTTAVPWNASWSNKSLEQIWNNMTWMEWDREINNYTSLIHSLIEESQNQQEKNEQELLELDKWASLWNWFNITNWLWYIKLFIMIVGGLVGLRIVFAVLSIVNRVRQGYSPLSFQTHLPNPRGPDRPEGIEEEGGERDRDRSIRLVNGSLALIWDDLRSLCLFSYHRLRDLLLIVTRIVELLGRRGWEALKYWWNLLQYWSQELKNSAVNLLNATAIAVAEGTDRVIELVQAAYRAIRHIPRRIRQGLERILL

SEQ ID NO: 19 - protein, 36 aa, Human immunodeficiency virus
  misc_feature: T-651
MTWMEWDREINNYTSLIHSLIEESQNQQEKNEQELL

SEQ ID NO: 20 - protein, 38 aa, Human immunodeficiency virus
  misc_feature: mutated amino acid sequence derived from the HIV-1 gp41 ectodomain (bp-position 621-656; T-2635)
TTWEAWDRAIAEYAARIEALIRAAQEQQEKNEAALREL

SEQ ID NO: 21 - protein, 123 aa, Human immunodeficiency virus
  misc_feature: HIV-1 gp41 ectodomain variant I568P (single mutant)
VQARQLLSGIVQQQNNLLRAIEGQQHLLQLTVWGPKQLQARILAVERYLKDQQLLGIWGCSGKLICTTAVPWNASWSNKSLEQIWNNMTWMEWDREINNYTSLIHSLIEESQNQQEKNEQELL

SEQ ID NO: 22 - protein, 132 aa, Human immunodeficiency virus
  misc_feature: HIV-1 gp41 ectodomain variant I568P, L550E, L566E, I580E (quadruple mutant)
MGAASMTLTVQARQLLSGIVQQQNNELRAIEGQQHLEQLTVWGPKQLQARELAVERYLKDQQLLGIWGCSGKLICTTAVPWNASWSNKSLEQIWNNMTWMEWDREINNYTSLIHSLIEESQNQQEKNEQELL

SEQ ID NO: 23 - DNA, 133 bp, Artificial
  Insert 4964
agatctatatatatatatgctagcgaaattgtgttgacacagtctccagccaccctgtctttgtctccaggggaaagagccaccctctcctgcagggccagtcagagtgttagtagctacttagcctggtacc

SEQ ID NO: 24 - DNA, 249 bp, Artificial
  Insert 4965
agatcttttgccaccatggacaccctgtgcagcaccctgctcctgctgaccatccccagctgggtgctctcccaaatctggaacaacatgacctggatggagtgggaccgcgagatcaataactacacaagcttgatccactctctgatcgaggaaagccagaaccagcaggagaagaacgagcaggagctcctgggcgggggtggatccggcggcgggggcagcggcgggggaggctccggcgctagc

SEQ ID NO: 25 - DNA, 279 bp, Artificial
  Insert 4966
agatcttttgccaccatggacaccctgtgcagcaccctgctcctgctgaccatccccagctgggtgctctcccaaatctggaacaacatgacctggatggagtgggaccgcgagatcaataactacacaagcttgatccactctctgatcgaggaaagccagaaccagcaggagaagaacgagcaggagctcctgggcgggggtggctccggcggcgggggcagcggcgggggaggctccggcgggggcggatccgggggcggtggcagcggcgctagc

SEQ ID NO: 26 - DNA, 252 bp, Artificial
  Insert 4967
agatcttttgccaccatggacaccctgtgcagcaccctgctcctgctgaccatccccagctgggtgctctcccaaatctggaacaacatgacctggatggagtgggaccgcgagatcaataactacacaagcttgatccactccctgatcgaggaaagccagaaccagcaggagaagaacgagcaggagctcctgggatccagctccagctccagctccagctccagcagtagctccagctctggcgctagc

SEQ ID NO: 27 - DNA, 292 bp, Artificial
  Insert 4968
agatctagaggaactgctcagttaggacccagagggaaccatgggcagccaggtgcacctcctgtccttcctcctgctgtggatcagcgacactcgagccgagaccacctgggaggcgtgggaccgcgccatcgccgagtacgccgctcgcatcgaagctttgatccgggccgcacaggagcagcaggagaagaacgaggctgcccttcgcgaactgggcgggggtggctccggcggcgggggcagcggcgggggcggatccggccgaactgtggctgcaccatctgtcttc

SEQ ID NO: 28 - DNA, 589 bp, Artificial
  Insert 4969
agatctagctctgggagaggagcccagcactagaagtcggcggtgtttccattcggtgatcagcactgaacacagaggactcaccatggagtttgggctgagctgggtgttcctcgtggcactgctcaggggtgtacagtgtcaggtgcaggcccgccagctgctctccggcatcgtccagcagcaaaacaatctgctgcgggcgatcgaggggcagcagcacctcctgcagctgacggtgtggggtcccaagcagctgcaggcccgcattctggccgtggaacggtacctgaaggaccagcagctgctcggcatctggggatgctctggcaagcttatctgcaccacagccgtcccctggaacgctagctggagtaacaaaagcctggagcaaatttggaacaacatgacctggatggagtgggatcgcgagatcaataattacacaagcctgatccactccctgatcgaggaaagccagaaccagcaggagaagaacgagcaggagctcctgggcgggggcggatccggcggcgggggcagcggtgggggcggctccggccgaactgtggctgcaccatctgtcttc

SEQ ID NO: 29 - DNA, 192 bp, Artificial
  Insert 4970
aagcttgtccccgggcaaaggcgggggcggcagcggcggcgggggatccggtgggggcggctccggcggcaacatgacctggatggagtgggatcgcgagatcaataattacacaagcctgatccactccctgatcgaggaaagccagaaccagcaggagaagaacgagcaggagctcctgtgagtgctagc

SEQ ID NO: 30 - DNA, 195 bp, Artificial
  Insert 4971
aagcttgtccccgggcaaaggatccagctccagctccagctccagctccagcagtagctccagctctggcaacaacatgacctggatggagtgggatcgcgagatcaataattacacaagcctgatccactccctgatcgaggaaagccagaaccagcaggagaagaacgagcaggagctcctgtgagtgctagc

SEQ ID NO: 31 - DNA, 195 bp, Artificial
  Insert 4972
aagcttgtccccgggcaaaggcgggggcggcagcggcggcgggggatccggtgggggcggctccggcggtaccacctgggaggcgtgggaccgcgccatcgccgagtacgccgctcgcatcgaagcgttgatccgggccgcacaggagcagcaggagaagaacgaggctgcccttcgcgaactgtgagtgctagc

SEQ ID NO: 32 - DNA, 195 bp, Artificial
  Insert 4973
aagcttgtccccgggcaaaggatccagctccagctccagctccagctccagcagtagctccagctctggtaccacctgggaggcgtgggaccgcgccatcgccgagtacgccgctcgcatcgaagcgttgatccgggccgcacaggagcagcaggagaagaacgaggctgcccttcgcgaactgtgagtgctagc

SEQ ID NO: 33 - DNA, 198 bp, Artificial
  Insert 4974
aagcttgtccccgggcaaaggccagcagcaacaggggcagcagcagcagggccagcaacagcagggtaacaacaccacctgggaggcgtgggaccgcgccatcgccgagtacgccgctcgcatcgaagcgttgatccgggccgcacaggagcagcaggagaagaacgaggctgcccttcgcgaactgtgagtgctagc

SEQ ID NO: 34 - DNA, 435 bp, Artificial
  Insert 4975
aagcttgtccccgggcaaagggagcaccatgggcgcggcctccatgacgctgaccgtgcaggcccgccagctgctctccggcatcgtccagcagcaaaacaatgagctgcgggcaattgaggggcagcagcacctcgaacagctgacggtgtggggtcccaagcagctgcaggcccgcgagctggccgtggaacggtacctgaaggaccagcagctgctcggcatctggggatgctctggcaagctgatctgcaccacagccgtcccctggaacgccagctggagtaacaaaagcctcgagcaaatttggaacaacatgacctggatggagtgggatcgcgagatcaataattacacaagcctgatccactccctgatcgaggaaagccagaaccagcaggagaagaacgagcaggagctcctgtgagtgctagc

SEQ ID NO: 35 - DNA, 230 bp, Artificial
  Insert 4978
aagcttcaacaggggagagtgtggcgggggtggctccggcggcgggggcagcggcgggggaggctccggcgggggcggatccgggggcggtggcagcggcggcaacatgacctggatggagtgggatcgcgagatcaataactacaccagcctgatccactctctgatcgaggaaagccagaaccagcaggagaagaacgagcaggagctcctgtagagggagaggcgcc

SEQ ID NO: 36 - DNA, 200 bp, Artificial
  Insert 4979
aagcttcaacaggggagagtgtggccagcagcaacaggggcagcagcagcagggccagcaacagcagggtaacaacatgacctggatggagtgggatcgcgagatcaataactacaccagcctgatccactctctgatcgaggaaagccagaaccagcaggagaagaacgagcaggagctcctgtagagggagaggcgcc

SEQ ID NO: 37 - protein, 51 aa, Artificial
  T-2324
QARQLLSGIVQQQNNLLRAIEAQQHLLQLTVWGIKQLQARILAVERYLKDQ

(19) United States
(12) Patent Application Publication
(10) Pub. No.: US 2007/0148180 A1
(43) Pub. Date: Jun. 28, 2007

(54) Peptide-immunoglobulin-conjugate

(76) Inventors: Stephan Fischer, Polling (DE); Erhard Kopetzki, Penzberg (DE); Suryanarayana Sankuratri, San Jose, CA (US); Ralf Schumacher, Penzberg (DE)

Correspondence Address: ROCHE PALO ALTO LLC;PATENT LAW DEPT. M/S A2-250, 3431 HILLVIEW AVENUE, PALO ALTO, CA, 94304, US

(21) Appl. No.: 11/583,547

(22) Filed: Oct. 19, 2006

(30) Foreign Application Priority Data
Oct. 21, 2005 (EP) 5023002.8

(51) Int. Cl. A61K 39/42; C07H 21/04; C12P 21/06; C07K 16/46

(52) U.S. Cl. 424/159.1; 424/178.1; 530/388.3; 530/391.1; 435/69.1; 435/328; 435/320.1; 536/23.53

(57) ABSTRACT

The current invention is related to peptide-immunoglobulin-conjugates in which at least two of the termini of the immunoglobulin polypeptide chains are conjugated to a peptide, whereby the peptides can be different, similar or identical. The conjugation is effected on the nucleic acid level.

### RELATED APPLICATIONS

This application claims priority from EP05023002.8, filed Oct. 21, 2005, incorporated herein by reference in full.

### FIELD OF THE INVENTION

The present invention relates to a peptide-immunoglobulin-conjugate, wherein two or more peptides are each conjugated to one terminus of a light or a heavy chain of an immunoglobulin. The peptides can be different, similar or identical on the amino acid level. The immunoglobulin chain to which the peptides are conjugated is not a functional immunoglobulin chain.

### BACKGROUND OF THE INVENTION

The infection of cells by the HIV virus is effected by a process in which the membrane of the cells to be infected and the viral membrane are fused. A general scheme for this process is proposed: The viral envelope glycoprotein complex (gp120/gp41) interacts with a cell surface receptor located on the membrane of the cell to be infected. The binding of gp120 to the CD4 receptor, in combination with a co-receptor such as CCR-5 or CXCR-4, causes a change in the conformation of the gp120/gp41 complex. In consequence of this conformational change the gp41 protein is able to insert into the membrane of the target cell. This insertion is the beginning of the membrane fusion process.

It is known that the amino acid sequence of the gp41 protein differs between the different HIV strains because of naturally occurring polymorphisms. But the same domain architecture can be recognized, precisely, a fusion signal, two heptad repeat domains (HR1, HR2) and a transmembrane domain (in N- to C-terminal direction). It is suggested that the fusion (or fusogenic) domain is participating in the insertion into and disintegration of the cell membrane. The HR regions are built up of multiple stretches comprising 7 amino acids (“heptad”) (see e.g. Shu, W., et al., Biochemistry 38 (1999) 5378-85). Beside the heptads one or more leucine zipper-like motifs are present. This composition results in the formation of a coiled coil structure of the gp41 protein, and of peptides derived from these domains. Coiled coils are in general oligomers consisting of two or more interacting helices.

Peptides with amino acid sequences deduced from the HR1 or HR2 domain of gp41 are effective in vitro and in vivo inhibitors of HIV uptake into cells (for peptide examples, see e.g. U.S. Pat. No. 5,464,933, U.S. Pat. No. 5,656,480, U.S. Pat. No. 6,258,782, U.S. Pat. No. 6,348,568, and U.S. Pat. No. 6,656,906). For example, T20 (also known as DP178, Fuzeon®, an HR2 peptide) and T651 (U.S. Pat. No. 6,479,055) are very potent inhibitors of HIV infection.

It has been attempted to enhance the efficacy of HR2 derived peptides with, for example, amino acid substitutions or chemical crosslinking (Sia, S. K., et al, Proc Natl Acad Sci USA 99 (2002) 14664-69; Otaka, A., et al, Angew. Chem. Int. Ed. 41 (2002) 2937-40).

The conjugation of peptides to certain molecules can change their pharmacokinetic properties, for example, the serum half life of such peptide conjugates can be increased. Conjugations are reported, for example, for: pegylated Interleukin-6 (EP 0 442 724); pegylated erythropoietin (WO 01/02017); chimeric molecules comprising endostatin and immunoglobulins (US 2005/008649); secreted antibody based fusion proteins (US 2002/147311); fusion polypeptides comprising albumin (US 2005/0100991; human serum albumin U.S. Pat. No. 5,876,969); pegylated polypeptides (US 2005/0114037); and for interferon fusions.

Fusions of polypeptides and immunoglobulins combine the antigen determining characteristics of an immunoglobulin with the biological activity of a polypeptide. Thus, the immunoglobulin part of the conjugate exhibits the targeting function and the polypeptide part provides the biological activity. This is reported, for example, for immunotoxins comprising Gelonin and an antibody (WO 94/26910); modified transferrin-antibody fusion proteins (US 2003/0226155); antibody-cytokine fusion proteins (US 2003/0049227); and fusion proteins consisting of a peptide with immuno-stimulatory, membrane transport, or homophilic activity and an antibody (US 2003/0103984).

In WO 2004/085505, long acting biologically active conjugates consisting of biologically active compounds chemically linked to macromolecules are reported.

### SUMMARY OF THE INVENTION

The object of the invention is to provide a peptide-immunoglobulin conjugate in which more than one peptide is conjugated to said immunoglobulin.

The invention comprises a peptide-immunoglobulin-conjugate in which the immunoglobulin consists of two heavy chains, or two heavy chains and two light chains, in which the immunoglobulin is a non-functional immunoglobulin, in which a peptide bond conjugates the carboxy-terminal amino acid of the immunoglobulin chain to the amino-terminal amino acid of the peptide or the carboxy-terminal amino acid of the peptide to the amino-terminal amino acid of the immunoglobulin chain, and in which the conjugate has the following general formula,
immunoglobulin-[peptide]ₙ
wherein n is an integer of from 2 to 8, wherein each peptide may be the same or different.

In one embodiment the peptide is a biologically active peptide.

In another embodiment the peptide consists of a peptidic linker and a biologically active peptide.

In one embodiment, the peptides of the conjugate all have an amino acid sequence identity of 90% or more with each other.

In still another embodiment, the immunoglobulin is an immunoglobulin of the G class (IgG) or E class (IgE).

In another embodiment, the biologically active polypeptide is an antifusogenic peptide.

In a further embodiment, the non-functional immunoglobulin is an immunoglobulin that binds any human antigen with a K_{D}-value of 10⁻⁵ mol/l or higher.

In a further embodiment, the non-functional immunoglobulin is an immunoglobulin a) in which both heavy and/or light chains lack a part or all of one or more framework or/and hypervariable regions, or b) in which both heavy and/or light chains have no variable region, or c) that has a binding affinity for human antigens of 10⁻⁵ mol/l or higher, or d) that has a binding affinity for human antigens of 10⁻⁵ mol/l or higher and a binding affinity for a non-human antigen of 10⁻⁷ mol/l or lower.

Further encompassed by the invention is a method for the production of a conjugate according to the invention, said method comprising cultivating a cell containing one or more expression vectors, which contain one or more nucleic acid molecules encoding the conjugate according to the invention, under conditions suitable for the expression of the conjugate and recovering the conjugate from the cell or the culture medium.

The invention also comprises pharmaceutical compositions, containing a conjugate according to the invention, or a pharmaceutically acceptable salt thereof together with a pharmaceutically acceptable excipient or carrier.

Further encompassed by the invention is the use of a conjugate according to the invention for the manufacture of a medicament for the treatment of viral infections.

In one embodiment, the viral infection is an HIV infection.

Another embodiment of the invention is a method for treating a patient with a conjugate according to the invention, comprising administering an effective amount of the conjugate of the invention to a patient in the need of an antiviral treatment.

### DESCRIPTION OF THE INVENTION

The invention comprises a peptide-immunoglobulin-conjugate in which the immunoglobulin consists essentially of two heavy chains or two heavy chains and two light chains, in which the immunoglobulin is non-functional, in which a peptide bond conjugates the carboxy-terminal amino acid of the immunoglobulin chain to the amino-terminal amino acid of the peptide or the carboxy-terminal amino acid of the peptide to the amino-terminal amino acid of the immunoglobulin chain, and in which the conjugate has the following general formula, wherein the position of the [peptide]-part in this general formula does not indicate the conjugation position at which the peptide is connected to the immunoglobulin
immunoglobulin-[peptide]ₙ
wherein n is an integer of from 2 to 8, and wherein each peptide may be the same or different.

Within the scope of the present invention some of the terms used are defined as follows:

A “gene” denotes a nucleic acid segment, e.g. on a chromosome or plasmid, which is necessary for the expression of a peptide, polypeptide or protein. Besides the coding region, the gene comprises other functional elements including a promoter, introns (in most cases), and a terminator.

A “structural gene” denotes the polypeptide-coding region of a gene, without any signal sequence.

An “antifusogenic peptide” is a peptide which inhibits events associated with membrane fusion or the membrane fusion event itself, including without limitation the inhibition of infection of uninfected cells by a virus due to membrane fusion. The antifusogenic peptides are preferably linear peptides. They can be derived, for example, from the gp41 ectodomain, such as DP107, and DP178. Examples of such peptides can be found in U.S. Pat. No. 5,464,933, U.S. Pat. No. 5,656,480, U.S. Pat. No. 6,013,263, U.S. Pat. No. 6,017,536, U.S. Pat. No. 6,020,459, U.S. Pat. No. 6,093,794, U.S. Pat. No. 6,060,065, U.S. Pat. No. 6,258,782, U.S. Pat. No. 6,348,568, U.S. Pat. No. 6,479,055, U.S. Pat. No. 6,656,906, WO 1996/19495, WO 1996/40191, WO 1999/59615, WO 2000/69902, and WO 2005/067960. For example, the amino acid sequences of such peptides comprise or can be selected from the group of SEQ ID NO: 1 to 10 of U.S. Pat. No. 5,464,933; SEQ ID NO: 1 to 15 of U.S. Pat. No. 5,656,480; SEQ ID NO: 1 to 10 and 16 to 83 of U.S. Pat. No. 6,013,263; SEQ ID NO: 1 to 10, 20 to 83 and 139 to 149 of U.S. Pat. No. 6,017,536; SEQ ID NO: 1 to 10, 17 to 83 and 210 to 214 of U.S. Pat. No. 6,093,794; SEQ ID NO: 1 to 10, 16 to 83 and 210 to 211 of U.S. Pat. No. 6,060,065; SEQ ID NO: 1286 and 1310 of U.S. Pat. No. 6,258,782; SEQ ID NO: 1129, 1278-1309, 1311 and 1433 of U.S. Pat. No. 6,348,568; SEQ ID NO: 1 to 10 and 210 to 238 of U.S. Pat. No. 6,479,055; SEQ ID NO: 1 to 171, 173 to 216, 218 to 219, 222 to 228, 231, 233 to 366, 372 to 398, 400 to 456, 458 to 498, 500 to 570, 572 to 620, 622 to 651, 653 to 736, 739 to 785, 787 to 811, 813 to 815, 816 to 823, 825, 827 to 863, 865 to 875, 877 to 883, 885, 887 to 890, 892 to 981, 986 to 999, 1001 to 1003, 1006 to 1018, 1022 to 1024, 1026 to 1028, 1030 to 1032, 1037 to 1076, 1078 to 1079, 1082 to 1117, 1120 to 1176, 1179 to 1213, 1218 to 1223, 1227 to 1237, 1244 to 1245, 1256 to 1268, 1271 to 1275, 1277, 1345 to 1348, 1350 to 1362, 1364, 1366, 1368, 1370, 1372, 1374 to 1376, 1378 to 1379, 1381 to 1385, 1412 to 1417, 1421 to 1426, 1428 to 1430, 1432, 1439 to 1542, 1670 to 1682, 1684 to 1709, 1712 to 1719, 1721 to 1753, 1755 to 1757 of U.S. Pat. No. 6,656,906; or SEQ ID NO: 5 to 95 of WO2005/067960, each of which is incorporated herein by reference. The antifusogenic peptide has an amino acid sequence of from about 5 to about 100 amino acids, preferably of from about 10 to about 75 amino acids, and more preferred of from about 15 to about 50 amino acids.

The term “biologically active molecule” as used herein refers to a biological macromolecule such as a peptide, protein, nucleoprotein, mucoprotein, lipoprotein, synthetic polypeptide or protein, and the like, that causes a biological effect when administered in artificial biological systems, such as bioassays using cell lines and viruses, or in vivo to an animal, including but not limited to birds and mammals, including humans. This biological effect can be, for example, enzyme inhibition, activation or allosteric modification, binding to a receptor, either at the binding site or circumferential, blocking or activating a receptor, signal triggering, and the like.

An “expression vector” is a nucleic acid molecule encoding a protein to be expressed in a host cell. Typically, an expression vector comprises a prokaryotic plasmid propagation unit, e.g., for E. coli, comprising an origin of replication, a selection marker, a eukaryotic selection marker, and one or more expression cassettes for the expression of the gene(s) of interest each comprising a promoter, a structural gene, and a transcription terminator including a polyadenylation signal. Gene expression is usually placed under the control of a promoter, and such a nucleic acid is said to be “operably linked” to the promoter. Similarly, a regulatory element and a core promoter are operably linked if the regulatory element modulates the activity of the core promoter.

A “polycistronic transcription unit” is a transcription unit in which more than one structural gene is under the control of the same promoter.

An “isolated peptide” is a polypeptide that is essentially free from contaminating cellular components, such as carbohydrate, lipid, or other proteinaceous impurities associated with the peptide in nature. Typically, a preparation of isolated peptide contains the peptide in a highly purified form, i.e. at least about 80% pure, at least about 90% pure, at least about 95% pure, greater than 95% pure, or greater than 99% pure. One way to show that a particular protein preparation contains an isolated peptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same peptide in alternative physical forms, such as dimers or alternatively glycosated or derivatized forms.

As used herein, the term “immunoglobulin” refers to a protein consisting of one or more polypeptides substantially encoded by immunoglobulin genes and variants or fragments thereof. The different polypeptides of which an immunoglobulin is composed of are referred to depending on their weight as light chain and as heavy chain. The recognized immunoglobulin genes include the different constant region genes as well as the myriad immunoglobulin variable region genes. Immunoglobulins may exist in a variety of formats. Each of the heavy and light chains contains a variable region (generally the amino terminal portion of the polypeptide chain). The variable domain of an immunoglobulin's light or heavy chain comprises different segments: four framework regions (FR) and three hypervariable regions (“complementarity determining regions”, or CDR). Each of the heavy and light polypeptide chains comprises a constant region (generally the carboxyl terminal portion of the polypeptide chain). The constant region of the heavy chain mediates, inter alia, the binding of the antibody a) to cells bearing a Fc-gamma receptor (FcγR), such as phagocytic cells, or b) to cells bearing the neonatal Fc receptor (FcRn) also known as Brambell receptor. It also mediates the binding to some factors including factors of the classical complement system such as component (C1q).

An immunoglobulin according to the present invention comprises at least two heavy chain polypeptides. Optionally, two light chain polypeptides may also be present. The immunoglobulins according to the invention are non-functional immunoglobulins.

The term “non-functional immunoglobulin” as used within this application denotes an immunoglobulin or immunoglobulin chain that binds to a human antigen with a K_{D}-value (binding affinity) of 10⁻⁵ mol/l or higher (e.g. 10⁻³ mol/l), preferably with a K_{D}-value of 10⁻⁴ mol/l or higher. A “human antigen” is an antigen derived from the human body, such as a human protein, carbohydrate, lipid, and the like. The binding affinity is determined with a standard binding assay, such as surface plasmon resonance technique (Biacore®). This binding affinity value need not be treated as an exact value: it is merely a point of reference. It is used to determine and/or select immunoglobulins that show essentially no immunoglobulin-typical specific target binding for human targets/antigens, and thus have no human therapeutic activity alone. For example, a non-functional immunoglobulin is an immunoglobulin that does not specifically bind any particular human antigen or epitope. At the same time, non-specific interactions, for example ionic interactions, may be present. This does not exclude that the immunoglobulin may show a specific target binding for non-human targets or antigens. This specific target binding of a non-human antigen is associated with a K_{D}-value of 10⁻⁷ mol/l or lower (e.g. 10⁻¹⁰ mol/l), preferably with a K_{D}-value of 10⁻⁸ mol/l or lower.

The term “linker” or “peptidic linker” as used within this application denotes peptide linkers of natural and/or synthetic origin. They are building up a linear amino acid chain wherein the 20 naturally occurring amino acids are the monomeric building blocks. The chain has a length of from 1 to about 50 amino acids, preferred between about 3 and about 25 amino acids. The linker may contain repetitive amino acid sequences or sequences of naturally occurring polypeptides, such as polypeptides with a hinge function. The linker has the function to ensure that a peptide conjugated to an immunoglobulin can perform its biological activity by allowing the peptide to fold correctly and to be presented properly.

Preferably the linker is a “synthetic peptidic linker” that is designed to be rich in glycine, glutamine and/or serine residues. These residues are arranged in small repetitive units of up to about five amino acids, such as GGGGS, QQQQG or SSSSG. This small repetitive unit may be repeated for about two to about five times to form a multimeric unit. At the amino- and/or carboxy-terminal ends of the multimeric unit, up to six additional arbitrary, naturally occurring amino acids may be added. Other synthetic peptidic linkers are composed of a single amino acid, that is repeated between 10 to 20 times, such as Serine in the linker SSSSSSSSSSSSSSS. At each of the amino- and/or carboxy-terminal end up to six additional arbitrary, naturally occurring amino acids may be present.

The term “amino acid” as used within this application denotes the group of naturally occurring carboxy α-amino acids comprising alanine (three letter code: ala, one letter code: A), arginine (arg, R), asparagine (asn, N), aspartic acid (asp, D), cysteine (cys, C), glutamine (gln, Q), glutamic acid (glu, E), glycine (gly, G), histidine (his, H), isoleucine (ile, I), leucine (leu, L), lysine (lys, K), methionine (met, M), phenylalanine (phe, F), proline (pro, P), serine (ser, S), threonine (thr, T), tryptophan (trp, W), tyrosine (tyr, Y), and valine (val, V).

Methods and techniques known to a person skilled in the art, which are useful for carrying out the current invention, are described e.g. in Ausubel, F. M., ed., Current Protocols in Molecular Biology, Volumes I to III (1997), Wiley and Sons; Sambrook et al., Molecular Cloning: A Laboratory Manual, Second Edition, Cold Spring Harbor Laboratory Press, Cold Spring Harbor, N.Y. (1989).

The current invention comprises an immunoglobulin conjugate in which at least two of the termini of the immunoglobulin are conjugated to a peptide. Immunoglobulins are assigned to five different classes: IgA (immunoglobulin of class A), IgD, IgE, IgG and IgM. Between these classes, the immunoglobulins differ in their overall structure. Looking at the building blocks similarities can be seen. All immunoglobulins are built up of pairs of polypeptide chains, comprising a so-called immunoglobulin light polypeptide chain (short: light chain) and a so-called immunoglobulin heavy polypeptide chain (short: heavy chain). The common structure of immunoglobulins of the IgG class is presented in FIG. 1.

In complex proteins, which are composed of different subunits, due to the modular structure more than one amino-terminus and more than one carboxy-terminus are available. The immunoglobulins of classes G and E, for example, possess each two pairs of heavy and light chains. Thus, four amino-termini and four carboxy-termini are present in each immunoglobulin molecule. This allows for a maximum number of eight peptides to be conjugated to an IgG or IgE.

The immunoglobulin provides the scaffold to which the peptides are connected by genetic means. Therefore also immunoglobulins having no functional variable domain or lacking all or a part of one or more variable domain regions and thus not possessing any antigen binding abilities can be employed in the current invention as non-functional immunoglobulin.

The peptide which is introduced at a terminus of an immunoglobulin chain is small of size compared to the entire immunoglobulin. For example, the smallest immunoglobulins, immunoglobulins of class G, have a molecular weight of approximately 150 kDa; a modification has a size of less than 12.5 kDa, which is equivalent to about 100 amino acids, in general less than 7.5 kDa, which is equivalent to about 60 amino acids.

The peptides are introduced into the immunoglobulin by molecular biological techniques on the nucleic acid level.

The peptide conjugated to the immunoglobulin has an amino acid sequence of from about 5 to about 100 amino acid residues, preferably of from about 10 to about 75 amino acid residues, more preferably of from about 15 to about 50 amino acid residues. The polypeptide conjugated to the immunoglobulin is selected from the group comprising biologically active molecules/peptides. These molecules cause a biological effect when administered to an artificial biological system, a living cell or living organism, such as birds or mammals, including humans. These biologically active compounds comprise, but are not limited to, agonists as well as antagonists of enzymes, receptors, immunoglobulins, and the like, targeted agents exhibiting cytotoxic, antiviral, antibacterial, or anti-cancer activity, as well as antigens. Preferably the biologically active peptides are selected from the group of antifusogenic peptides. The immunoglobulin conjugates of the current invention are useful for pharmaceutical, therapeutic, or diagnostic applications.

The biologically active peptide can be selected from, without limitation, the group consisting of hedgehog proteins, bone morphogenetic proteins, growth factors, erythropoietin, thrombopoietin, G-CSF, interleukins and interferons, protein hormones, antiviral peptides, antifusogenic peptides, antiangiogenic peptides, cytotoxic peptides and the like.

For the terminal conjugation of more than one peptide to an immunoglobulin different distributions exist. The number of peptides, which can be conjugated to an immunoglobulin, is from one to the combined number of amino- and carboxy-termini of the immunoglobulin polypeptide chains.

If a single peptide is conjugated to an immunoglobulin, the peptide can occupy any one of the termini of the immunoglobulin. Likewise, if the maximum possible number of peptides is conjugated to an immunoglobulin, all termini are occupied by a single peptide. If the number of peptides which are conjugated to the immunoglobulin is larger than one but smaller than the maximum possible number, different distributions of the peptides at the termini of the immunoglobulin are possible.

For example, if four peptides are conjugated to an immunoglobulin of the G or E class, five different combinations are possible (see Table 1). In two combinations all termini of one kind, i.e. all four amino-termini or all four carboxy-termini of the immunoglobulin chains, are conjugated to one peptide. The other termini are not conjugated. This results in one embodiment in an allocation of the modifications/conjugations in one area of the immunoglobulin. In the other cases the polypeptides are conjugated to a number of both termini. Within these combinations the conjugated peptides are allocated to different areas of the immunoglobulin. In either case, the sum of conjugated termini is four.

TABLE 1
Possible combination for the conjugation of four peptides to the
termini of an immunoglobulin composed of four polypeptide chains.

| number of occupied | number of occupied | total number of |
|---|---|---|
| amino-termini | carboxy-termini | occupied termini |
| 4 | 0 | 4 |
| 3 | 1 | 4 |
| 2 | 2 | 4 |
| 1 | 3 | 4 |
| 0 | 4 | 4 |

The current invention comprises immunoglobulins in which at least two of the termini are conjugated to a peptide. The conjugated peptide itself is not derived from an immunoglobulin. The amino acid sequences of the conjugated peptides can be different, similar or identical. In general, the amino acid sequences are different, i.e., they possess an amino acid identity of less than 90%. In one embodiment the amino acid sequence identity is in the range of from 90% to less than 100%; these amino acid sequences and the corresponding peptides are defined as similar. In another embodiment, the peptides have identical amino acid sequences.

Although the conjugated peptides may display a certain degree of homology or identity, they may also differ in the total length of their amino acid sequence.

The conjugation between the peptide and the immunoglobulin is performed on the nucleic acid level. Therefore a peptide bond between two amino acids conjugates the peptide and the immunoglobulin. Thus either the carboxy-terminal amino acid of the peptide is conjugated to the amino-terminal amino acid of the immunoglobulin chain or the carboxy-terminal amino acid of the immunoglobulin chain is conjugated to the amino-terminal amino acid of the peptide.

A further characteristic of the peptide-immunoglobulin conjugate according to the invention is that the complete conjugate is encoded by one or more nucleic acid molecules, preferably by two to eight nucleic acid molecules. This enables for the recombinant production of the immunoglobulin conjugate.

For the recombinant production of the peptide-immunoglobulin-conjugate according to the invention two or more nucleic acid molecules encoding different polypeptides are required, preferably two to eight nucleic acid molecules. These nucleic acid molecules encode the different immunoglobulin polypeptide chains of the conjugate and are referred to as structural genes. They can be part of the same expression cassette, or can alternatively be located in different expression cassettes. The assembly of the conjugate preferably takes place before the secretion of the conjugate, i.e., within the expressing cells. Therefore the nucleic acids molecules encoding the polypeptide chains of the conjugate are preferably co-expressed in the same host cell.

Generally speaking, for the production of unconjugated immunoglobulins two structural genes, one encoding the light chain and one encoding the heavy chain, are required. For the production of certain peptide-immunoglobulin-conjugates, additional structural genes encoding the conjugated immunoglobulin light and/or heavy chains are required. An example is displayed in FIG. 12, wherein all peptide-immunoglobulin-conjugates of an immunoglobulin and two different peptides are shown.

An immunoglobulin according to the invention that is composed of two heavy chains conjugated to identical peptides is encoded by one or two structural genes. In the case that both peptides are conjugated to the same terminal amino acid of the heavy chain, only one structural gene is employed. In case that one peptide is conjugated to the amino-terminal amino acid of the first heavy chain and the other peptide is conjugated to the carboxy-terminal amino acid of the second heavy chain, two structural genes are employed.

A further example is a conjugate in which both amino termini of the light chains are conjugated to different or similar peptides. In this case, three structural genes are required (FIG. 11). One structural gene encodes the unconjugated heavy chain (structural gene 2), one structural gene encodes the first light chain conjugated to peptide 1 (structural gene 1), and one structural gene encodes the second light chain conjugated to peptide 2 (structural gene 3). For the structural genes, one or more expression cassettes are designed which are located on one or more expression plasmids. In case that in the before example the conjugated peptides are identical only two structural genes are required, i.e. one encoding the not conjugated heavy chain and one encoding the amino-conjugated light chain (see FIG. 11: structural genes 1 and 3 are identical). All three building blocks of the peptide-immunoglobulin-conjugate are preferably expressed in the same cell. Assuming statistical assembly of the immunoglobulin chains, four different immunoglobulin conjugates can be realized. Of these, immunoglobulin 2 and immunoglobulin 2a are identical, and thus three different immunoglobulin conjugates are secreted into the medium. If all three structural genes are expressed stochiometrically, the ratio between immunoglobulin 1, immunoglobulin 2, and immunoglobulin 3 is 1:2:1. By enhancing or decreasing the expression of one or more of these structural genes the ratio of the assembled conjugates can be shifted to a preferred conjugate (1, 2 or 3). Methods therefore are known to a person skilled in the art using, for example, promoters with different promoter strength. In the case of identical peptides, only one immunoglobulin conjugate is formed and secreted into the culture medium.

The mixture of immunoglobulin conjugates obtained can be separated and purified by methods known to a person skilled in the art. These methods are well established and wide-spread used for immunoglobulin purification and are employed either alone or in combination. Such methods include, for example, affinity chromatography using microbial-derived proteins (e.g. protein A or protein G affinity chromatography), ion exchange chromatography (e.g. cation exchange (carboxymethyl resins), anion exchange (amino ethyl resins) and mixed-mode exchange), thiophilic adsorption (e.g. with beta-mercaptoethanol and other SH ligands), hydrophobic interaction or aromatic adsorption chromatography (e.g. with phenyl-Sepharose, aza-arenophilic resins, or m-aminophenylboronic acid), metal chelate affinity chromatography (e.g. with Ni(II)- and Cu(II)-affinity material), size exclusion chromatography, and preparative electrophoretical methods (such as gel electrophoresis, capillary electrophoresis) (Vijayalakshmi, M. A. Appl. Biochem. Biotech. 75 (1998) 93-102).

FIGS. 13 and 14 show the different conjugates, which can be obtained using a first peptide conjugated to the amino-terminus of a light chain and a second peptide conjugated to the carboxy-terminus of a heavy chain. In this case ten different immunoglobulin conjugates are generated starting with four different structural genes.

The peptide-immunoglobulin-conjugates show improved pharmacokinetics compared to the unconjugated peptides, such as serum half-life. At the same time, it is possible to increase the local concentration of the conjugated peptide with the peptide-immunoglobulin-conjugate according to the invention, because the conjugated peptides are presented and fixed in close vicinity by the immunoglobulin to which they are conjugated. It is also possible to provide more than one, i.e. two or more, different peptides conjugated to the same immunoglobulin.

The characteristics of the immunoglobulin conjugate of the current invention depend on the biological activity of the conjugated peptides. Therefore the peptides must adopt their natural three dimensional structure to be able to interact with their target, and should be presented properly without access restrictions. To prevent steric interference, the conjugated peptide may consist of a peptidic linker and a biologically active peptide (for peptidic linkers see Table 2).

TABLE 2
Peptidic linker.

| | | |
|---|---|---|
| | | SEQ |
| linker | linker nucleotide or amino acid | ID |
| number | sequence | NO: |

| | | |
|---|---|---|
| 1 | [Ser(Gly)₄]₃ | 01 |
| 2 | [Ser(Gly)₄]₅ | 02 |
| 3 | [Gly(Gln)₄]₃Gly | 03 |
| 4 | [Gly(Gln)₄]₃ | 04 |
| 5 | Gly(Ser)₁₅Gly | 05 |
| 6 | GST | 06 |
| 7 | [(Gly)₄Ser]₃-Gly-Ala-Ser | 07 |
| 8 | Gly(Ser)₁₅-Gly-Ala-Ser | 08 |
| 9 | [(Gly)₄Ser]₃-Gly | 09 |
| 10 | [(Gly)₄Ser]₅-Gly | 10 |
| 11 | [(Gly)₄Ser]₃-Gly₂ | 11 |
| 12 | [(Gly)₄Ser]₅-Gly₂ | 12 |
| 13 | agatcttttgccaccgctagc | 13 |
| 14 | aagcttgtccccgggcaaatgagtgctagc | 14 |
| 15 | agatctatatatatatatgctagc | 15 |
| 16 | ArgThrValAlaAlaProSerValPheIlePhe | 16 |
| 17 | aagcttcaacaggggagagtgttgaagggagaggcgcc | 17 |

All peptidic linkers can be encoded by a nucleic acid molecule and therefore can be recombinantly expressed. As the linkers are themselves peptides, the biologically active peptide is connected to the linker via a peptide bond that is formed between two amino acids. The peptidic linker is introduced between the biologically active peptide and the immunoglobulin chain to which the biologically active peptide is to be conjugated. Therefore three possible sequences in amino- to carboxy-terminal direction exist: a) biologically active peptide-peptidic linker-immunoglobulin polypeptide chain, b) immunoglobulin polypeptide chain-peptidic linker-biologically active peptide, or c) biologically active peptide-peptidic linker-immunoglobulin polypeptide chain-peptidic linker-biologically active peptide, whereby the biologically active peptide may be the same or different, and whereby the peptidic linker may be present or not, i.e., possible sequences in C- to N-terminal direction include d) biologically active peptide-immunoglobulin polypeptide chain, or e) immunoglobulin polypeptide chain-biologically active peptide, or f) biologically active peptide-immunoglobulin polypeptide chain-biologically active peptide, or combinations thereof, such as g) biologically active peptide-peptidic liner-immunoglobulin polypeptide chain-biologically active peptide.

With recombinant engineering methods known to a person skilled in the art, the immunoglobulin conjugates can be tailor-made on the nucleic acid/gene level. The nucleic acid sequences encoding immunoglobulins are known and can be obtained for example from genomic databases. Likewise the nucleic acid sequences of biologically active peptides are known or can easily be deduced from its amino acid sequence on the basis of the nucleotide triplet codons encoding the amino acids of the amino acid sequence of the biologically active peptide.

The elements required for the construction of an expression plasmid for the expression of the conjugate of the current invention are an expression cassette for the immunoglobulin light chain in its natural and/or modified and/or conjugated version, an expression cassette for the immunoglobulin heavy chain in its natural and/or modified and/or conjugated version, a selection marker, and an E. coli replication as well as selection unit. These cassettes comprise a promoter, the structural gene, a DNA segment encoding a secretion signal sequence, and a terminator. These elements are assembled in an operatively linked form either on one plasmid encoding all chains of the immunoglobulin conjugate, or on two or more plasmids each encoding one or more chains of the immunoglobulin conjugate.

For the expression of the encoded polypeptides the plasmid(s) is (are) introduced into a suitable host cell. Proteins are preferably produced in mammalian cells such as CHO cells, NS0, cells, Sp2/0 cells, COS cells, HEK cells, K562 cells, BHK cells, PER.C6 cells, and the like. The regulatory elements of the vector have to be selected in a way that they are functional in the selected host cell.

For the expression the host cell containing the plasmid encoding one or more chains of the immunoglobulin conjugate is cultivated under conditions suitable for the expression of the chains. The expressed immunoglobulin chains are functionally assembled. The fully processed peptide-immunoglobulin-conjugate is secreted into the medium.

The immunoglobulin part of the conjugate provides a scaffold to which the peptides are attached. The immunoglobulin is a non-functional immunoglobulin, i.e. it binds human antigens with a K_{D}-value (binding affinity) of 10⁻⁵ mol/l or higher (e.g. 10⁻³ mol/l). Immunoglobulins that fall within this definition are e.g. immunoglobulins in which both heavy and/or light chains lack a part or all of one or more framework or/and hypervariable regions, immunoglobulins in which both heavy and/or light chains have no variable region, immunoglobulins that have a K_{D}-value of 10⁻⁷ mol/l or lower (e.g. 10⁻¹⁰ mol/l) for a non-human antigen.

The following examples, sequence listing and figures are provided to aid the understanding of the present invention, the true scope of which is set forth in the appended claims. It is understood that modifications can be made in the procedures set forth without departing from the spirit of the invention.

### DESCRIPTION OF THE FIGURES

FIG. 1 Common structure of immunoglobulins of the IgG class.

FIG. 2 Plasmid map of the anti-IGF-1R γ1-heavy chain expression vector 4818.

FIG. 3 Plasmid map of the anti-IGF-1R κ-light chain expression vector 4802.

FIG. 4 Plasmid map of the γ1-heavy chain constant region gene vector 4962.

FIG. 5 Plasmid map of the modified anti-IGF-1R γ1-heavy chain expression vector 4961.

FIG. 6 Plasmid map of the modified anti-IGF-1R κ-light chain expression vector 4964.

FIG. 7 Plasmid map of the modified anti-IGF-1R light chain expression vector 4963.

FIG. 8 Coomassie Blue stained SDS-PAGE-gels of affinity purified immunoglobulin conjugates; sample arrangement according to table 6.

FIG. 9 Immunodetection of the light chain in cell culture supernatants after transient expression in HEK293 EBNA cells; sample arrangement according to table 6.

FIG. 10 Immunodetection of the heavy chain in cell culture supernatants after transient expression in HEK293 EBNA cells; sample arrangement according to table 6.

FIG. 11 Peptide-immunoglobulin-conjugates in which both amino termini of the light chains are conjugated to different or similar peptides.

FIG. 12 Peptide-immunoglobulin-conjugates consisting of an immunoglobulin and two different peptides.

FIGS. 13 and 14 Peptide-immunoglobulin-conjugates in which a first peptide is conjugated to the amino-terminus of a light chain of an immunoglobulin and a second peptide is conjugated to the carboxy-terminus of a heavy chain of an immunoglobulin.

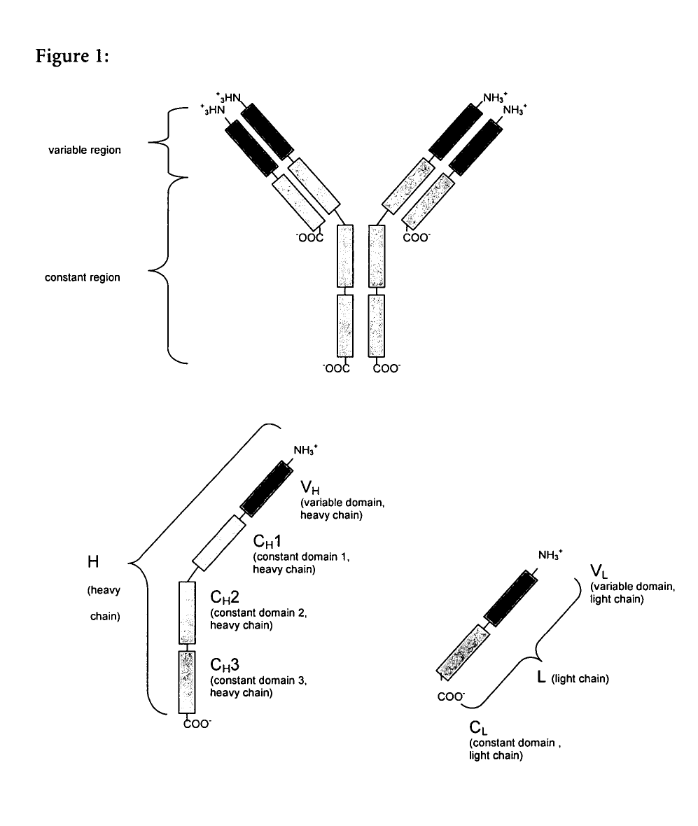

### EXAMPLES

Materials & Methods

General information regarding the nucleotide sequences of human immunoglobulins light and heavy chains is given in: Kabat, E. A. et al., (1991) Sequences of Proteins of Immunological Interest, Fifth Ed., NIH Publication No 91-3242.

Amino acids of antibody chains are numbered according to EU numbering (Edelman, G. M., et al., Proc Natl Acad Sci USA 63 (1969) 78-85; Kabat, E. A., et al., (1991) Sequences of Proteins of Immunological Interest, Fifth Ed., NIH Publication No 91-3242).

Recombinant DNA Techniques

Standard methods were used to manipulate DNA as described in Sambrook, J. et al., Molecular cloning: A laboratory manual; Cold Spring Harbor Laboratory Press, Cold Spring Harbor, N.Y., 1989. The molecular biological reagents were used according to the manufacturer's instructions.

Protein Determination

The protein concentration of the peptide-immunoglobulin-conjugate was determined by determining the optical density (OD) at 280 nm, using the molar extinction coefficient calculated on the basis of the amino acid sequence.

DNA Sequence Determination

DNA sequences were determined by double strand sequencing performed at MediGenomix GmbH (Martinsried, Germany).

DNA and Protein Sequence Analysis and Sequence Data Management

The GCG's (Genetics Computer Group, Madison, Wis.) software package version 10.2 and Infomax's Vector NTI Advance suite version 8.0 was used for sequence creation, mapping, analysis, annotation and illustration.

Gene Synthesis

Desired gene segments were prepared by Medigenomix GmbH (Martinsried, Germany) from oligonucleotides made by chemical synthesis. The 100-600 bp long gene segments which are flanked by singular restriction endonuclease cleavage sites were assembled by annealing and ligation of oligonucleotides including PCR amplification and subsequently cloned into the pCR2.1-TOPO-TA cloning vector (Invitrogen) via A-overhangs. The DNA sequence of the subcloned gene fragments were confirmed by DNA sequencing.

Affinity Purification of Immunoglobulin Conjugates

The expressed and secreted peptide-immunoglobulin-conjugates were purified by affinity chromatography using Protein A-Sepharose™ CL-4B (Amersham Bioscience) according to known methods. Briefly, after centrifugation (10,000×g for 10 minutes) and filtration through a 0.45 μm filter, the immunoglobulin conjugate containing clarified culture supernatants were applied on a Protein A-Sepharose™ CL-4B column equilibrated with PBS buffer (10 mM Na₂HPO₄, 1 mM KH₂PO₄, 137 mM NaCl and 2.7 mM KCl, pH 7.4). Unbound proteins were washed out with PBS equilibration buffer and 0.1 M citrate buffer, pH 5.5. The immunoglobulin conjugates were eluted with 0.1 M citrate buffer, pH 3.0, and the immunoglobulin conjugate containing fractions were neutralized with 1 M Tris-Base. Then, the immunoglobulin conjugates were extensively dialyzed against PBS buffer at 4° C., concentrated with an ultrafree centrifugal filter device equipped with a Biomax-SK membrane (Millipore) and stored in an ice-water bath at 0° C.

### Example 1

### Making of the Expression Plasmids

The gene segments encoding an insulin-like growth factor 1 receptor (IGF-1R) antibody (also referred to as anti-IGF-1R or 1R in the following) light chain variable region (V_{L}) and the human kappa-light chain constant region (C_{L}) (for sequences see US 2005/0008642) were joined as were gene segments for the anti-IGF-1R heavy chain variable region (V_{H}) and the human gamma1-heavy chain constant region (C_{H}1-Hinge-C_{H}2-C_{H}3).

a) Vector 4818

Vector 4818 is the expression plasmid for the transient expression of anti-IGF-1R antibody heavy chain (genomically organized expression cassette; exon-intron organization) in HEK293 EBNA cells. It comprises the following functional elements:

Beside the anti-IGF-1R gamma1-heavy chain expression cassette this vector contains:
a hygromycin resistance gene as a selectable marker,
an origin of replication, oriP, of Epstein-Barr virus (EBV),
an origin of replication from the vector pUC18 which allows replication of this plasmid in E. coli, and
a beta-lactamase gene which confers ampicillin resistance in E. coli.

The transcription unit of the anti-IGF-1R γ1-heavy gene is composed of the following elements:
the immediate early enhancer and promoter from the human cytomegalovirus (HCMV),
a synthetic 5′-untranslated region (UT),
a murine immunoglobulin heavy chain signal sequence including a signal sequence intron (signal seqeunce 1, intron, signal sequence 2 [L1-intron-L2]),
the cloned anti-IGF-1R variable heavy chain encoding segment arranged with a unique BsmI restriction site at the 5′-end (L2 signal sequence) and a splice donor site and a unique NotI restriction site at the 3′-end,
a mouse/human heavy chain hybrid intron 2 including the mouse heavy chain enhancer element (part JH₃, JH₄) (Neuberger, M. S., EMBO J. 2 (1983) 1373-78),
the genomic human γ1-heavy gene constant region,
the human γ1-immunoglobulin polyadenylation (“poly A”) signal sequence, and
the unique restriction sites AscI and SgrAI at the 5′- and 3′-end, respectively.

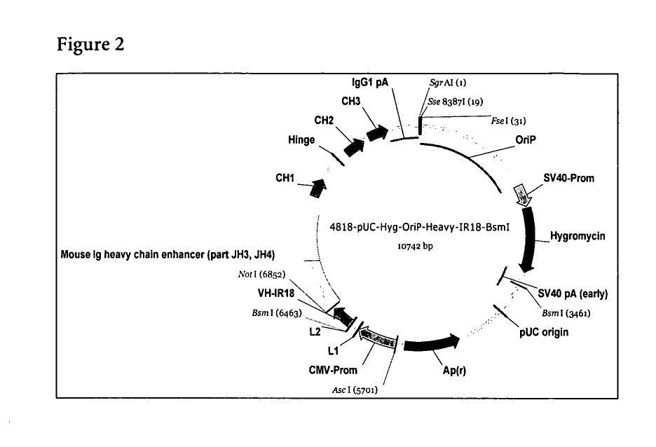

The plasmid map of the anti-IGF-1R γ1-heavy chain expression vector 4818 is shown in FIG. 2.

b) Vector 4802

Vector 4802 is the expression plasmid for the transient expression of anti-IGF-1R antibody light chain (cDNA) in HEK293 EBNA cells. It comprises the following functional elements.

Beside the anti-IGF-1R kappa-light chain expression cassette this vector contains:
a hygromycin resistance gene as a selectable marker,
an origin of replication, oriP, of Epstein-Barr virus (EBV),
an origin of replication from the vector pUC18 which allows replication of this plasmid in E. coli, and
a β-lactamase gene which confers ampicillin resistance in E. coli.

The transcription unit of the anti-IGF-1R κ-light gene is composed of the following elements:
the immediate early enhancer and promoter from the human cytomegalovirus (HCMV),
the cloned anti-IGF-1R variable light chain cDNA including
the native 5′-UT and
the native light chain signal sequence of the human immunoglobulin germline gene arranged with a unique BglII restriction site at the 5′-end,
the human κ-light gene constant region,
the human immunoglobulin κ-polyadenylation (“poly A”) signal sequence, and
the unique restriction sites AscI and FseI at the 5′- and 3′-end, respectively.

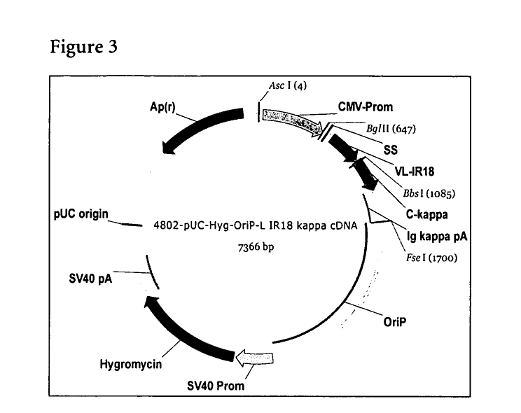

The plasmid map of the anti-IGF-1R κ-light chain expression vector 4802 is shown in FIG. 3.

c) Plasmid 4962

Vector 4962 served as basic structure for the assembling of expression plasmids 4965, 4966 and 4967. These plasmids enabled the transient expression of modified antibody heavy chains (N-terminal conjugation without variable domain, cDNA organization) in HEK 293 EBNA cells. Plasmid 4962 comprises the following functional elements.

Beside the expression cassette for the gamma1-heavy chain constant region this vector contains:
a hygromycin resistance gene as a selectable marker,
an origin of replication, oriP, of Epstein-Barr virus (EBV),
an origin of replication from the vector pUC18 which allows replication of this plasmid in E. coli, and
a beta-lactamase gene which confers ampicillin resistance in E. coli.

The transcription unit of the γ1-heavy chain constant region gene (C_{H}1-Hinge-C_{H}2-C_{H}3) is composed of the following elements:
the immediate early enhancer and promoter from the human cytomegalovirus (HCMV),

a synthetic linker (SEQ ID NO: 13) comprising a single BglII restriction site at the 5′-end and a single NheI restriction site at the 3′-end (NheI site within the C_{H1} N-terminus)

```
HCMV-promoter AlaSer (CH1)
              . . . agatcttttgccaccgctagc . . .
                          BglII NheI
```

the human γ1-heavy chain gene constant region (C_{H}1-Hinge-C_{H}2-C_{H}3, cDNA organization),
the human γ1-immunoglobulin polyadenylation (“poly A”) signal sequence, and
the unique restriction sites AscI and FseI at the 5′- and 3′-end, respectively.

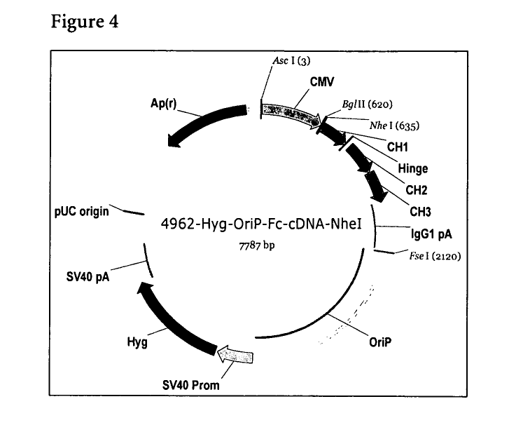

The plasmid map of the γ1-heavy chain constant region gene vector 4962 is shown in FIG. 4.

d) Plasmid 4961

Vector 4961 served as basic structure for the assembling of expression plasmids 4970 to 4975. These plasmids enabled the transient expression of modified antibody heavy chains (C-terminal conjugation) in HEK 293 EBNA cells.

Basic vector 4961 is the expression plasmid for the transient expression of anti-IGF-1R antibody heavy chain (genomically organized expression cassette) in HEK293 EBNA cells. It comprises the following functional elements.

Beside the anti-IGF-1R γ1-heavy chain expression cassette this vector contains:
a hygromycin resistance gene as a selectable marker,
an origin of replication, oriP, of Epstein-Barr virus (EBV),
an origin of replication from the vector pUC18 which allows replication of this plasmid in E. coli, and
a β-lactamase gene which confers ampicillin resistance in E. coli.

The transcription unit of the anti-IGF-1R γ1-heavy gene is composed of the following elements:
the immediate early enhancer and promoter from the human cytomegalovirus (HCMV),
a synthetic 5′-UT,
a murine immunoglobulin heavy chain signal sequence including a signal sequence intron (L1, intron, L2),
the cloned anti-IGF-1R variable heavy chain encoding segment arranged with a unique BsmI restriction site at the 5′-(L2 signal sequence) and a splice donor site and a unique NotI restriction site at the 3′-end,
a mouse/human heavy chain hybrid intron 2 including the mouse heavy chain enhancer element (part JH₃, JH₄) (Neuberger, M. S., EMBO J. 2 (1983) 1373-78),

the genomic human γ1-heavy gene constant region and a slightly modified C_{H}3-IgG₁ polyadenylation (pA) joining region (SEQ ID NO: 14, insertion of a unique HindIII and NheI restriction site).

```
            CH3 IgGl pA
. . . ctaagcttgtccccgggcaaaTGAgtgctagcgccggcaagcc . . .
. . . LeuSerLeuSerProGlyLys
                HindIII NheI
```

the human γ1-immunoglobulin polyadenylation (“poly A”) signal sequence, and
the unique restriction sites AscI and FseI at the 5′- and 3′-end, respectively.

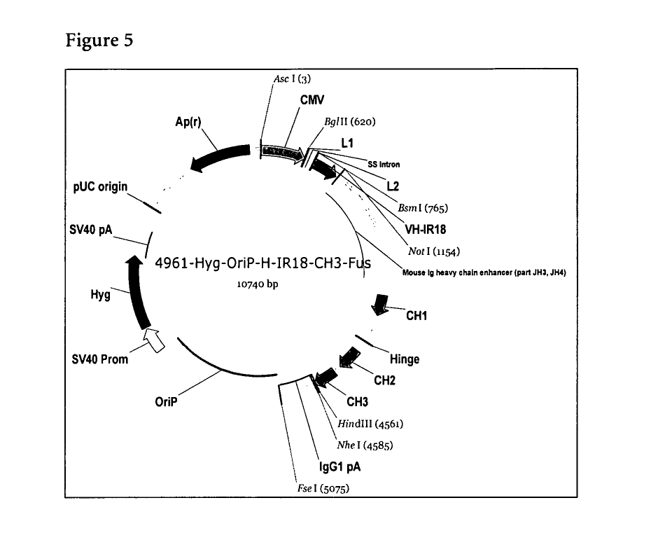

The plasmid map of the modified anti-IGF-1R γ1-heavy chain expression vector 4961 is shown in FIG. 5.

e) Plasmid 4964

Vector 4964 served as basic structure for the assembling of expression plasmids 4976 and 4977. These plasmids enabled the transient expression of modified anti-IGF-1R antibody light chains (N-terminal conjugation) in HEK 293 EBNA cells.

The plasmid 4964 is a variant of expression plasmid 4802.

The transcription unit of the anti-IGF-1R κ-light gene was modified as indicated below:

The native light chain signal sequence is replaced by a synthetic linker arranged with a unique BglII restriction site at the 5′- and a unique NheI restriction site at the 3′-end directly joined to the V_{L}-region (SEQ ID NO: 15).

```
                                                              |- V_{L}-IGF-IR
. . . agatctatatatatatatgctagcgaaattgtgttgaca . . .
                                                AlaSerGluIleValLeuThr . . .
BglII NheI
```

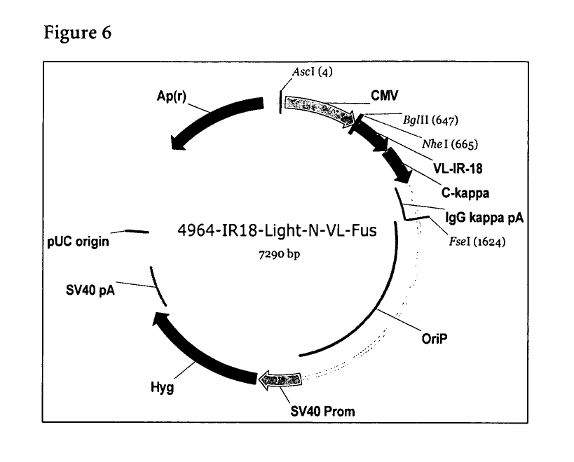

The plasmid map of the modified anti-IGF-1R κ-light chain expression vector 4964 is shown in FIG. 6.

f) Plasmid 4969

The expression plasmids 4968 and 4969 are derived from plasmid 4802 which is an expression plasmid for the anti-IGF-1R antibody light chain. The plasmid encodes a modified antibody light chain fragment (N-terminal conjugation without variable domain; polypeptide-linker-constant region of kappa chain).

For the construction of plasmids 4968 and 4969 a unique BglII restriction site was introduced at the 3′-end of the CMV-promoter and a unique BbsI restriction site was introduced inside of the constant region of anti-IGF-1R antibody light chain (SEQ ID NO: 16).

|-- C-kappa BbsI
cgaactgtggctgcaccatctgtcttcatcttc . . .
ArgThrValAlaAlaProSerValPheIlePhe . . .

g) Plasmid 4963

Vector 4963 served as basic structure for the assembling of expression plasmids 4978 and 4979. These plasmids enabled the transient expression of modified anti-IGF-1R antibody light chains (C-terminal conjugation) in HEK 293 EBNA cells.

The plasmid 4963 is a variant of expression plasmid 4802.

The transcription unit of the anti-IGF-1R κ-light gene was modified as indicated below:

the human κ-light chain constant gene region was slightly modified at the C-kappa-Ig-kappa pA joining region (insertion of a unique HindIII and KasI restriction site, SEQ ID NO: 17).

. . . C-kappa Ig-kappa-pA
. . . AaaagcttcaacaggggagagtgtTGAagggagaggcgccccca
. . . LysSerPheAsnArgGlyGluCys
HindIII Ka SI

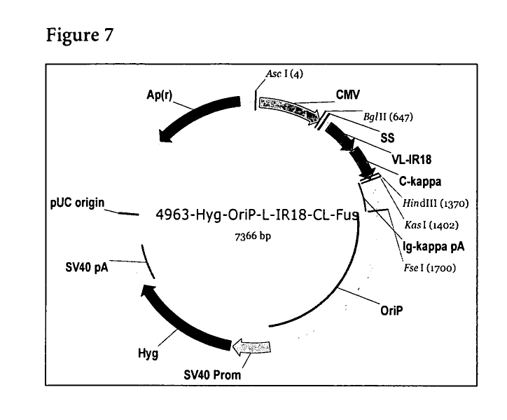

The plasmid map of the modified anti-IGF-1R light chain expression vector 4963 is shown in FIG. 7.

### Example 2

### Making the Final Expression Plasmids

The immunoglobulin fusion genes (heavy and light chain) comprising the immunoglobulin gene segment, optional linker gene segment and polypeptide gene segment have been assembled with known recombinant methods and techniques by connection of the according nucleic acid segments.

The nucleic acid sequences encoding the peptidic linkers and polypeptides were each synthesized by chemical synthesis and then ligated into an E. coli plasmid. The subcloned nucleic acid sequences were verified by DNA sequencing.

The employed immunoglobulin polypeptide chains (full length heavy or light chain), respectively, the immunoglobulin polypeptide chain fragments (constant region of antibody light or heavy chain), the location of the polypeptide conjugation (N- or C-terminal), the employed linkers and the employed polypeptides are listed in Table 2 (on page 13), Table 3 and Table 3a.

TABLE 3
Employed proteins and polypeptides; the amino acid sequence and
the numbering of the positions is as in the BH8 reference strain
(Locus HIVH3BH8; HIV-1 isolate LAI/IIIB clone BH8 from France;
Ratner, L. et al., Nature 313 (1985) 277-84).

| proteins and polypeptides | SEQ ID NO: |
|---|---|
| HIV-1 gp41 | 18 |
| (position 507-851 of BH8 gp 160) | |
| T-651 (see e.g. U.S. Pat. No. 6,656,906) | 19 |
| T-2635 | 20 |
| (see e.g. WO 2004/029074) | |
| HIV-1 gp41 ectodomain variant | 21 |
| single mutant: I568P | |
| HIV-1 gp41 ectodomain variant | 22 |
| quadruple mutant: I568P, L550E, | |
| L566E, I580E | |

TABLE 3a
Chemically prepared gene segments used for immunoglobulin
conjugate gene construction.

| Insert | SEQ ID NO: |
|---|---|
| Insert 4964 (introduction of | 23 |
| unique restriction sites) | |
| Insert 4965 (with T-651) | 24 |
| Insert 4966 (with T-651) | 25 |
| Insert 4967 (with T-651) | 26 |
| Insert 4968 (with T-2635) | 27 |
| Insert 4969 (gp41 single mutant) | 28 |
| Insert 4970 (with T-651) | 29 |
| Insert 4971 (with T-651) | 30 |
| Insert 4972 (with T-2635) | 31 |
| Insert 4973 (with T-2635) | 32 |
| Insert 4974 (with T-2635) | 33 |
| Insert 4975 | 34 |
| (gp41 quadruple mutant) | |
| Insert 4978 (with T-651) | 35 |
| Insert 4979 (with T-651) | 36 |

The components used for the construction of the final expression plasmids for transient expression of the modified immunoglobulin polypeptide light and heavy chains (the expression cassettes) are listed in Table 4 with respect to the used basis plasmid, cloning site, and inserted nucleic acid sequence encoding the conjugated immunoglobulin polypeptides.

TABLE 4
Components employed in the construction of the used expression
plasmids.

| | | | |
|---|---|---|---|
| Expression | Basis | Inserted DNA | Cloning |
| plasmid | vector | gene segment | sites |

N-terminal conjugation: Heavy chain (without variable domain)

| | | | |
|---|---|---|---|
| 4965 | 4962 | Insert 4965 (249 Bp) | BglII/NheI |
| 4966 | 4962 | Insert 4966 (279 Bp) | BglII/NheI |
| 4967 | 4962 | Insert 4967 (252 Bp) | BglII/NheI |

N-terminal conjugation: Light chain (without variable domain)

| | | | |
|---|---|---|---|
| 4968 | 4802 | Insert 4968 (292 Bp) | BglII/BbsI |
| 4969 | 4802 | Insert 4969 (589 Bp) | BglII/BbsI |

C-terminal conjugation: Heavy chain

| | | | |
|---|---|---|---|
| 4970 | 4961 | Insert 4970 (192 Bp) | HindIII/NheI |
| 4971 | 4961 | Insert 4971 (195 Bp) | HindIII/NheI |
| 4972 | 4961 | Insert 4972 (195 Bp) | HindIII/NheI |
| 4973 | 4961 | Insert 4973 (195 Bp) | HindIII/NheI |
| 4974 | 4961 | Insert 4974 (198 Bp) | HindIII/NheI |
| 4975 | 4961 | Insert 4975 (435 Bp) | HindIII/NheI |

C-terminal conjugation: Light chain

| | | | |
|---|---|---|---|
| 4978 | 4963 | Insert 4978 (230 Bp) | HindIII/KasI |
| 4979 | 4963 | Insert 4979 (200 Bp) | HindIII/KasI |

N-terminal conjugation: Light chain (including the variable domain)

| | | | |
|---|---|---|---|
| 4976 | 4964 | Insert 4965 (249 Bp) | HindIII/KasI |
| 4977 | 4964 | Insert 4967 (252 Bp) | HindIII/KasI |

In Table 5 is listed: the used polypeptides with HIV-1 inhibitory properties (T-651, T-2635 and HIV-1 gp41 ectodomain variants), the used peptidic linkers to join the immunoglobulin light or heavy chain with the biologically active peptide, and the deduced molecular weight of the modified antibody chains as deduced from the encoded amino acid sequences.

TABLE 5
Summary of the employed polypeptides and the deduced molecular
weight of the modified immunoglobulin polypeptide chains.

| | | | |
|---|---|---|---|
| | | | peptidic |
| expression | | molecular | linker |
| plasmid | polypeptide | weight [Da] | SEQ ID NO: |

Reference plasmids

| | | | |
|---|---|---|---|
| 4818 | heavy chain | 49263.5 | no linker |
| 4802 | light chain | 23572.2 | no linker |

N-terminal fusions: Heavy chain (without variable domain)

| | | | |
|---|---|---|---|
| 4965 | T-651 | 42227.3 | 09 |
| 4966 | T-651 | 42857.9 | 10 |
| 4967 | T-651 | 42644.7 | 05 |

N-terminal fusions: Light chain (without variable domain)

| | | | |
|---|---|---|---|
| 4968 | T-2635 | 17294.9 | 09 |
| 4969 | Gp41 single | 27247.3 | 09 |
| | mutant | | |

C-terminal fusions: Heavy chain

| | | | |
|---|---|---|---|
| 4970 | T-651 | 54900.5 | 11 |
| 4971 | T-651 | 55374.9 | 05 |
| 4972 | T-2635 | 54322.9 | 11 |
| 4973 | T-2635 | 55081.7 | 05 |
| 4974 | T-2635 | 55655.4 | 03 |
| 4975 | Gp41 quadruple | 64771.9 | 06 |
| | mutant | | |

C-terminal fusions: Light chain

| | | | |
|---|---|---|---|
| 4978 | T-651 | 29839.7 | 12 |
| 4979 | T-651 | 30029.1 | 03 |

N-terminal fusions: Light chain (including the variable domain)

| | | | |
|---|---|---|---|
| 4976 | T-651 | 29851.9 | 07 |
| 4977 | T-651 | 30269.2 | 08 |

### Example 3

### Transient Expression of Immunoglobulin Variants in HEK293 EBNA Cells

Recombinant immunoglobulin variants were generated by transient transfection of adherent growing HEK293-EBNA cells (human embryonic kidney cell line 293 expressing Epstein-Barr Virus nuclear antigen; American type culture collection deposit number ATCC # CRL-10852) cultivated in DMEM (Dulbecco's modified Eagle's medium, Gibco) supplemented with 10% ultra-low IgG FCS (fetal calf serum, Gibco), 2 mM Glutamine (Gibco), 1% volume by volume (v/v) nonessential amino acids (Gibco) and 250 μg/ml G418 (Roche Molecular Biochemicals). For transfection, Fugene™ 6 Transfection Reagent (Roche Molecular Biochemicals) was used in a ratio of reagent (μl) to DNA (μg) ranging from 3:1 to 6:1. Immunoglobulin light and heavy chains were expressed from two different plasmids using a molar ratio of light chain to heavy chain encoding plasmid from 1:2 to 2:1. Immunoglobulin variants containing cell culture supernatants were harvested at days 4 to 11 after transfection. Supernatants were stored at 0° C. in an ice-water bath until purification.

General information regarding the recombinant expression of human immunoglobulins in e.g. HEK293 cells is given in: Meissner, P. et al., Biotechnol. Bioeng. 75 (2001) 197-203.

### Example 4

### Expression Analysis Using SDS PAGE, Western Blotting Transfer and Detection with Immunoglobulin Specific Antibody Conjugates

The expressed and secreted peptide-immunoglobulin-conjugates were processed by sodium dodecyl sulfate (SDS) polyacrylamide gel electrophoresis (SDS-PAGE), and the separated immunoglobulin conjugate chains were transferred to a membrane from the gel and subsequently detected by an immunological method.

SDS-PAGE

LDS sample buffer, fourfold concentrate (4×): 4 g glycerol, 0.682 g Tris-Base, 0.666 g Tris-hydrochloride, 0.8 g LDS (lithium dodecyl sulfate), 0.006 g EDTA (ethylene diamine tetraacetic acid), 0.75 ml of a 1% by weight (w/w) solution of Serva Blue G250 in water, 0.75 ml of a 1% by weight (w/w) solution of phenol red, add water to make a total volume of 10 ml.

The culture broth containing the secreted peptide-immunoglobulin-conjugates was centrifuged to remove cells and cell debris. An aliquot of the clarified supernatant was admixed with ¼ volumes (v/v) of 4×LDS sample buffer and 1/10 volume (v/v) of 0.5 M 1,4-dithiotreitol (DTT). Then the samples were incubated for 10 min. at 70° C. and protein separated by SDS-PAGE. The NuPAGE® Pre-Cast gel system (Invitrogen) was used according to the manufacturer's instruction. In particular, 10% NuPAGE® Novex® Bis-Tris Pre-Cast gels (pH 6.4) and a NuPAGE® MOPS running buffer was used.

Western Blot

Transfer buffer: 39 mM glycine, 48 mM Tris-hydrochloride, 0.04% by weight (w/w) SDS, and 20% by volume methanol (v/v)

After SDS-PAGE the separated immunoglobulin conjugate polypeptide chains were transferred electrophoretically to a nitrocellulose filter membrane (pore size: 0.45 μm) according to the “Semidry-Blotting-Method” of Burnette (Burnette, W. N., Anal. Biochem. 112 (1981) 195-203).

Immunological Detection

TBS-buffer: 50 mM Tris-hydrochloride, 150 mM NaCl, adjusted to pH 7.5

Blocking solution: 1% (w/v) Western Blocking Reagent (Roche Molecular Biochemicals) in TBS-buffer

TBST-Buffer: 1×TBS-buffer with 0.05% by volume (v/v) Tween-20

For immunological detection the western blotting membranes were incubated with shaking at room temperature two times for 5 minutes in TBS-buffer and once for 90 minutes in blocking solution.

Detection of the Immunoglobulin Conjugate Polypeptide Chains

Heavy chain: For detection of the heavy chain of the peptide-immunoglobulin-conjugate a purified rabbit anti-human IgG antibody conjugated to a peroxidase was used (DAKO, Code No. P 0214).

Light chain: The light chain of the peptide-immunoglobulin-conjugate was detected with a purified peroxidase conjugated rabbit anti-human kappa light chain antibody (DAKO, Code No. P 0129).

For visualization of the antibody light and heavy chains washed and blocked Western blot membranes were first incubated in case of a heavy chain with a purified rabbit anti-human IgG antibody conjugated to a peroxidase or in case of a light chain with a purified peroxidase conjugated rabbit anti-human kappa light chain antibody in a 1:10,000 dilution in 10 ml blocking solution at 4° C. with shaking over night. After washing the membranes three times with TBTS-buffer and once with TBS buffer for 10 min. at room temperature, the Western-blot membranes were developed with a Luminol/peroxid-solution generating chemiluminescence (Lumi-Light^{PLUS} Western Blotting Substrate, Roche Molecular Biochemicals). Therefore the membranes were incubated in 10 ml Luminol/peroxid-solution for 10 seconds to 5 minutes and the emitted light was detected afterwards with a Lumi-Imager F1 Analysator (Roche Molecular Biochemicals) and/or was recorded with an x-ray-film.

The intensity of the spots was quantified with the LumiAnalyst Software (Version 3.1).

Multiple-Staining of Immunoblots

The secondary peroxidase-labeled antibody conjugate used for the detection can be removed from the stained blot by incubating the membrane for one hour at 70° C. in 1 M Tris-hydrochloride-buffer (pH 6.7) containing 100 mM beta-mercaptoethanol and 20% (w/v) SDS. After this treatment the blot can be stained with a different secondary antibody a second time. Prior to the second detection the blot is washed three times at room temperature with shaking in TBS-buffer for 10 minutes each.

The sample arrangement is listed in tables 6a to 6c.

TABLE 6a
Sample arrangement of SDS PAGE gels/Western blots - gel 1

| | | Expression plasmids | | |
|---|---|---|---|---|
| Slot | Sample | Light chain | Heavy chain | Note |
| 1 | molecular weight (MW) | | | |
| | marker | | | |
| 2 | reference unconjugated | | | |
| | immunoglobulin, 50 ng | | | |
| 3 | reference unconjugated | | | |
| | immunoglobulin, 150 ng | | | |
| 4 | reference unconjugated | | | |
| | immunoglobulin, 500 ng | | | |
| 5 | HEK293 culture medium | | | |
| 6 | 3 | 4802 (wt) | 4818 (wt) | control |
| 7 | 4 | 4802 (wt) | 4961 (wt) | control |
| 8 | 5 | 4963 (wt) | 4818 (wt) | control |
| 9 | 6 | 4802 (wt) | 4965 | N-term; heavy; without VH |
| 10 | 7 | 4802 (wt) | 4966 | N-term; heavy; without VH |
| 11 | 8 | 4802 (wt) | 4967 | N-term; heavy; without VH |
| 12 | 9 | 4968 | 4818 (wt) | N-term; light; without VL |
| 13 | 10 | 4969 | 4818 (wt) | N-term; light; without VL |
| 14 | 11 | 4802 (wt) | 4970 | C-term; heavy |
| 15 | 12 | 4802 (wt) | 4971 | C-term; heavy |

TABLE 6b
Sample arrangement of SDS PAGE gels/Western blots - gel 2

| | | Expression plasmids | | |
|---|---|---|---|---|
| Slot | Sample | Light chain | Heavy chain | Note |
| 1 | MW marker | | | |
| 2 | reference | | | control |
| | unconjugated | | | |
| | immunoglobulin, | | | |
| | 100 ng | | | |
| 3 | HEK293 culture | | | |
| | medium | | | |
| 4 | 13 | 4802 (wt) | 4972 | C-term; heavy |
| 5 | 14 | 4802 (wt) | 4973 | C-term; heavy |
| 6 | 15 | 4802 (wt) | 4974 | C-term; heavy |
| 7 | 16 | 4802 (wt) | 4975 | C-term; heavy |
| 8 | 17 | 4976 | 4818 (wt) | N-term; light |
| 9 | 18 | 4977 | 4918 (wt) | N-term; light |
| 10 | 19 | 4978 | 4918 (wt) | C-term; light |
| 11 | 20 | 4979 | 4918 (wt) | C-term; light |
| 12 | 21 | 4978 | 4970 | C-term; light |
| | | | | C-term; heavy |

TABLE 6c
Sample arrangement of SDS PAGE gels/Western blots - gel 3

| | | Expression plasmids | | |
|---|---|---|---|---|
| Slot | Sample | Light chain | Heavy chain | Note |
| 1 | MW marker | | | |
| 2 | reference unconjugated | | | control |
| | immunoglobulin, 100 ng | | | |
| 3 | HEK293 culture medium | | | |
| 4 | 22 | 4979 | 4971 | C-term; light |
| | | | | C-term; heavy |
| 5 | 23 | 4979 | 4973 | C-term; light |
| | | | | C-term; heavy |
| 6 | 24 | 4968 | 4965 | N-term; light; without VL |
| | | | | C-term; heavy; without VH |
| 7 | 25 | 4969 | 4966 | N-term; light; without VL |
| | | | | N-term; heavy; without VH |
| 8 | 26 | 4976 | 4972 | N-term; light |
| | | | | C-term; heavy |
| 9 | 27 | 4977 | 4973 | N-term; light |
| | | | | C-term; heavy |
| 10 | 28 | 4977 | 4974 | N-term; light |
| | | | | C-term; heavy |
| 11 | 29 | 4976 | 4966 | N-term; light; |
| | | | | N-term; heavy; without VH |
| 12 | 30 | 4977 | 4967 | N-term; light |
| | | | | N-term; heavy; without VH |
| 13 | 31 | 4969 | 4975 | C-term; light; without VL |
| | | | | C-term; heavy |

### EXAMPLE 5

### Detection of the Assembled Immunoglobulin Conjugates

Purification and Concentration of Immunoglobulin Conjugate Polypeptides by Affinity Binding to Protein A Sepharose™ CL-4B

HEK 293 EBNA cells containing one or more plasmids were cultivated under conditions suitable for the transient expression of the structural immunoglobulin polypeptide chain gene(s) located on the plasmid(s) for 6 to 10 days. To 1 ml clarified culture supernatant in a 1.8 ml Eppendorf cup, 0.1 ml of a Protein A-Sepharose™ CL-4B (Amersham Biosciences) suspension (1:1 (v/v) suspension of Protein A-Sepharose™ in PBS buffer (10 mM Na₂HPO₄, 1 mM KH₂PO₄, 137 mM NaCl and 2.7 mM KCl, pH 7.4)) was added. The suspension was incubated for a time of between 1 and 16 hours at room temperature with shaking. Thereafter, the Sepharose beads were sedimented by centrifugation (30 s, 5000 rpm) and the supernatant was discarded. The Sepharose pellet was washed subsequently each with 1.6 ml PBS buffer, 1.6 ml 0.1 M citrate buffer pH 5.0 and 1.6 ml distilled water. The Protein A-bound immunoglobulin was extracted from the Sepharose beads with 0.1 ml 1×LDS-PAGE sample buffer at 70° C. for 5 to 10 min. The analysis was done by SDS-PAGE separation and staining with Coomassie brilliant blue as described in example 4.

Results:

Expression/Secretion-analysis of heavy and light chains after transient expression:

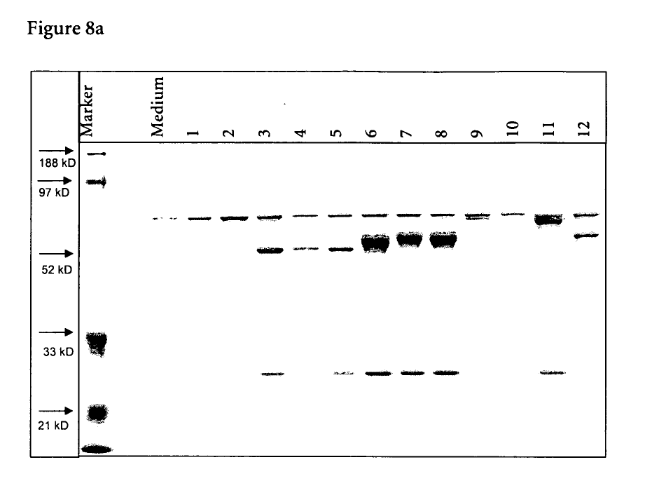

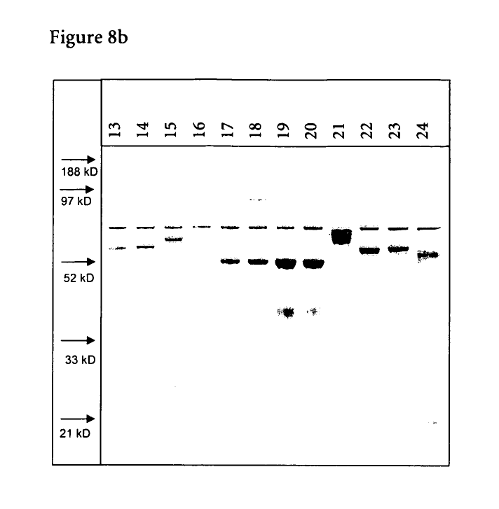

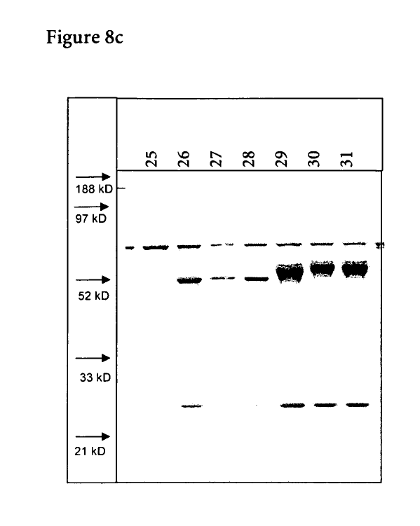

FIG. 8a-c: Coomassie Blue stained SDS-PAGE-gels of affinity purified immunoglobulin conjugates; sample arrangement according to table 6.

Immunodetection of immunoglobulin polypeptide chains:

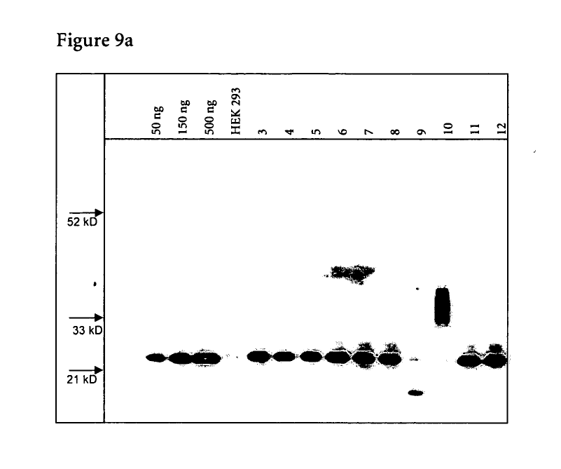

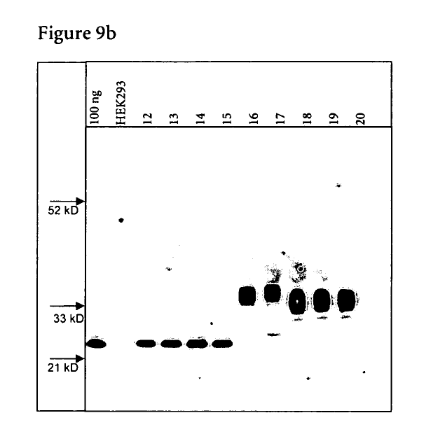

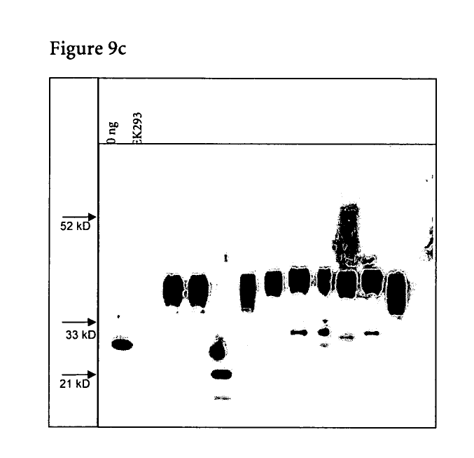

FIG. 9a-c: Immunodetection of the light chain in cell culture supernatants after transient expression in HEK293 EBNA cells.

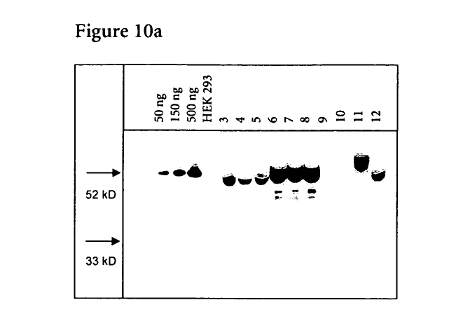

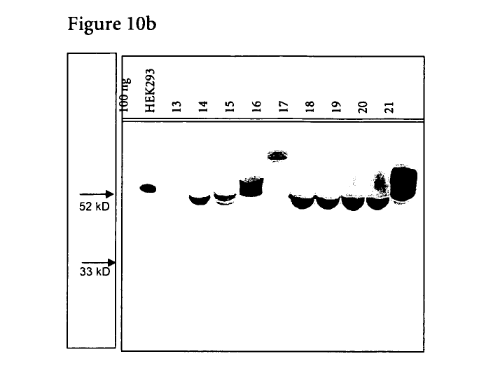

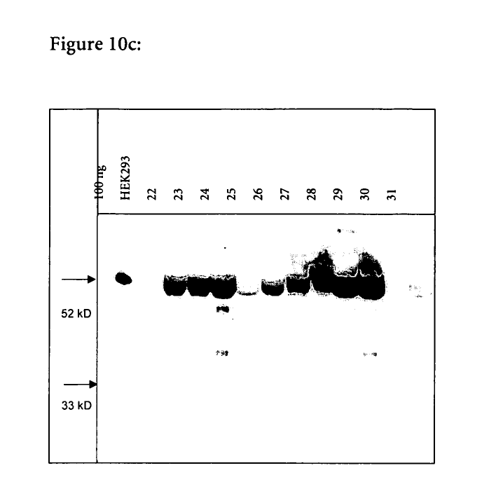

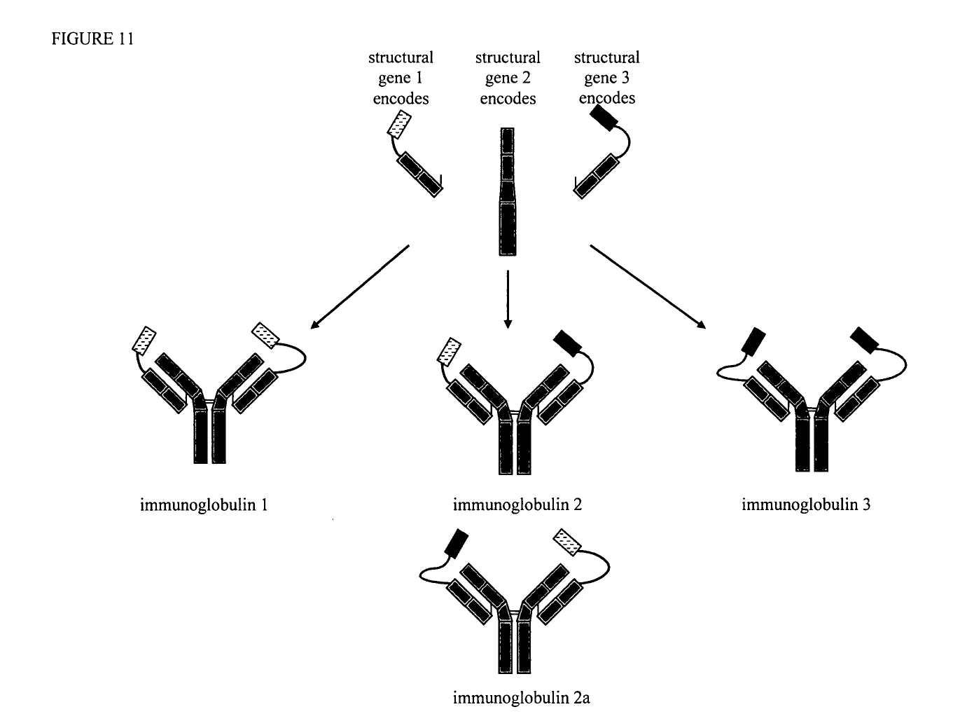

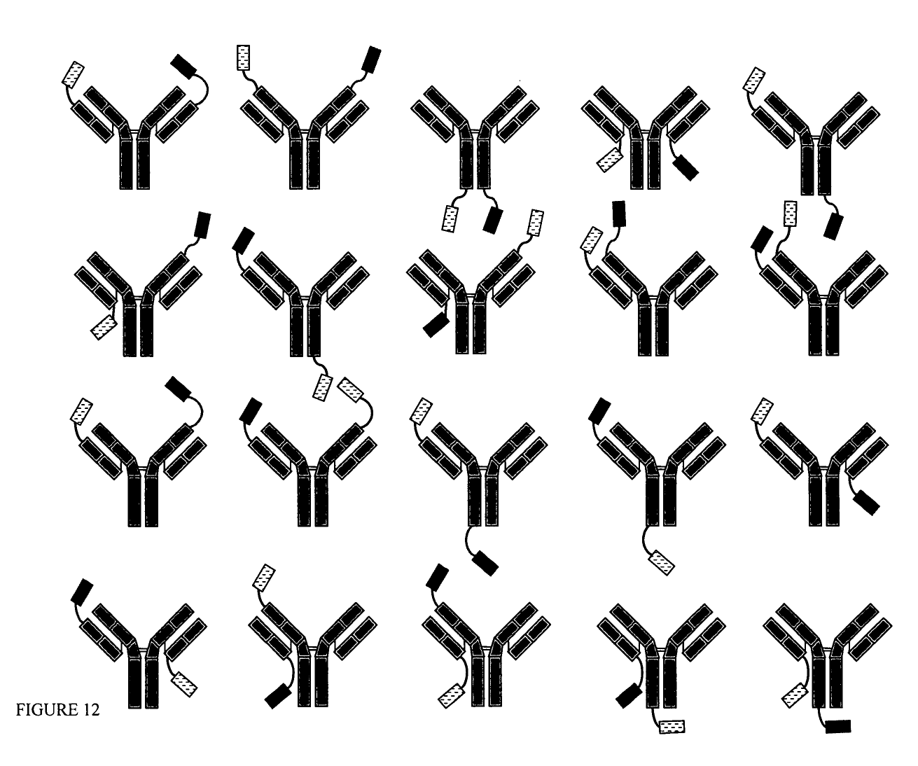

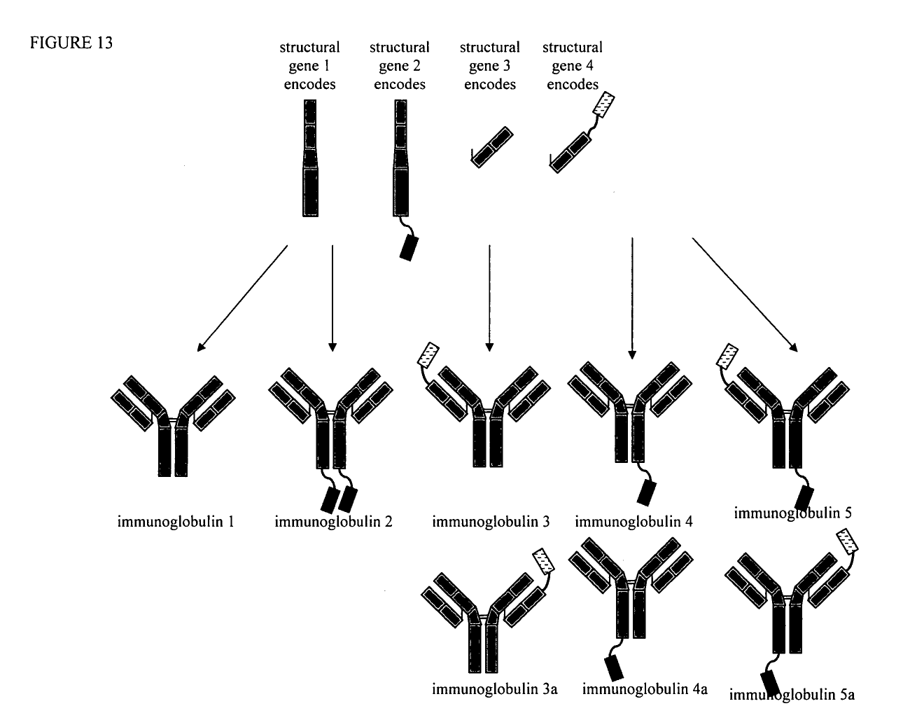

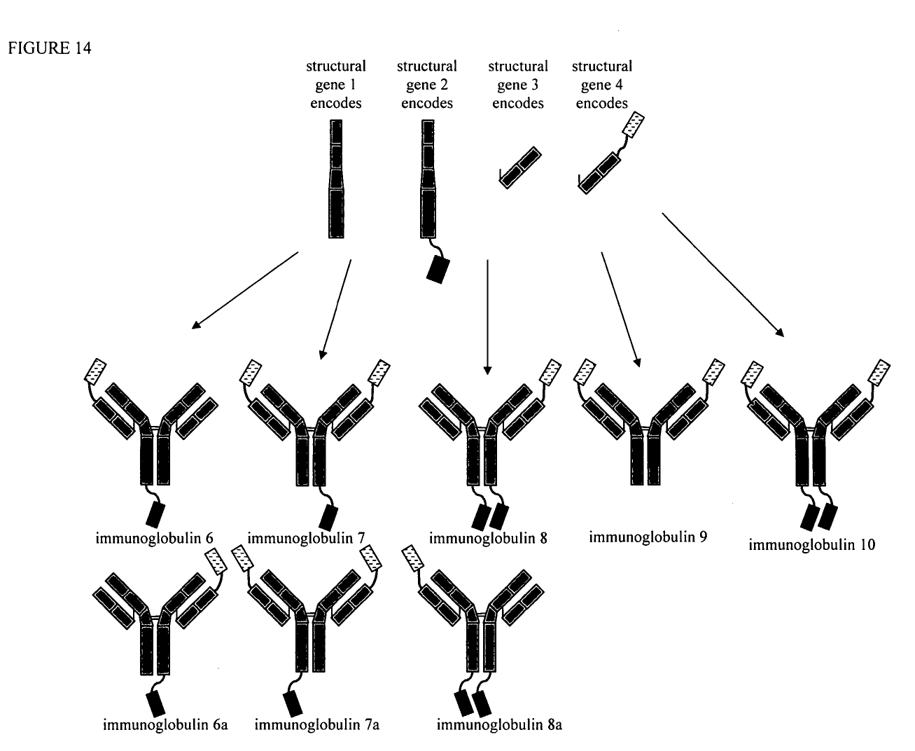

FIG. 10a-c: Immunodetection of the heavy chain in cell culture supernatants after transient expression in HEK293 EBNA cells.

From FIG. 8a-c, 9a-c and 10a-c it can be deduced that the immunoglobulin light and heavy chains are transiently expressed and secreted into the culture medium. In the case that the immunoglobulin chain possesses one or several glycosylation sites the final peptide-immunoglobulin-conjugate and the single immunoglobulin conjugate chains respectively have no exactly defined molecular weight but a molecular weight distribution depending on the extent of glycosylation. This causes in SDS-PAGE that the species all representing one immunoglobulin conjugate chain do not migrate homogeneously and thus the bands are broadened.

Because affinity binding of an immunoglobulin to Protein A is based only on an interaction of the Fc-part of the heavy chain(s), and because in addition to the heavy chain a light chain was detected after the SDS-PAGE and staining with Coomassie dye, it can be concluded that the immunoglobulin conjugates are correctly assembled and are consisting of light and heavy chains.

### Example 6

### Quantitation of the Expressed Heavy Chains with Human IgG ELISA

The immunoglobulin conjugate heavy chain polypeptide concentration in cell culture supernatants was determined by a sandwich ELISA which used a biotinylated anti-human IgG F(ab′)₂ fragment as the capture reagent and for detection a peroxidase-conjugated anti-human IgG F(ab′)₂ antibody fragment.

Streptavidin coated 96-well plates (Pierce Reacti-Bind™ Streptavidin Coated Polystyrene Strip Plates, Code No. 15121) were coated with 0.5 μg/ml biotinylated goat polyclonal anti-human IgG F(ab′)₂ antibody fragment ((F(ab′)₂<h-Fcγ>Bi; Dianova, Code No. 109-066-098) capture antibody (0.1 ml/well) in diluent buffer (diluent buffer: PBS buffer containing 0.5% weight by volume (w/v) bovine serum albumin) by incubation for one hour at room temperature (RT) under shaking. Thereafter, the plates were washed three times with more than 0.3 ml wash buffer (wash buffer: PBS containing 1% weight by volume (w/v) Tween 20). IgG immunoglobulin conjugate containing cell culture supernatants (samples) were diluted serially (twofold) up to a concentration of 0.5-20 ng/ml in diluent buffer, added to plates and incubated for one hour at RT with shaking. Purified anti-IGF-1R standard antibody (0.5-20 ng/ml) in diluent buffer was used for the generation of an IgG protein standard curve. After washing the plates three times with 0.3 ml/well wash buffer, bound complexes to human Fcγ were detected with a peroxidase-conjugated F(ab′)₂ fragment of goat polyclonal anti-human F(ab′)₂-specific IgG (F(ab′)₂<h-Fcγ>POD; Dianova, Code No. 109-036-098). After washing the plates 3× with 0.3 ml/well wash buffer, the plates were developed with ABTS® (2,2′-azino-bis(3-ethylbenzthiazoline-6-sulfonic acid) peroxidase substrate solution (Roche Molecular Biochemicals, Code No. 1684302). After 10-30 minutes the absorbance was measured at 405 nm and 490 nm against a reagent blank (incubation buffer+ABTS solution) on a Tecan Spectrafluorplus plate reader (Tecan Deutschland GmbH). For background correction the absorbance at 490 nm was subtracted from the absorbance at 405 nm according to formula I. All samples were assayed at least as duplicates, and the values from double or triple absorbance measurements were averaged. The IgG content of the samples were calculated from a standard curve.
ΔA=(Aₛₐₘₚₗₑ⁴⁰⁵−Aₛₐₘₚₗₑ⁴⁹⁰)−(A_{blank}⁴⁰⁵−A_{blank}⁴⁹⁰) Formula I

### Example 7

### Live Virus Antiviral Assay

For the production of live NL-Bal viruses, plasmid pNL-Bal (US NIH Aids Reagent Program) was transfected into the HEK 293FT cell line (Invitrogen) cultured in Dulbecco's modified minimum medium (DMEM) containing 10% fetal calf serum (FCS), 100 U/mL Penicillin, 100 μg/mL Streptomycin, 2 mM L-glutamine and 0.5 mg/mL geniticin (all media from Invitrogen/Gibco). The supernatants containing viral particles were harvested two days following transfection, and cellular debris was removed by filtration through a 0.45 μm pore size PES (polyethersulfon) filter (Nalgene) and stored at −80° C. in aliquots. For normalization in assay performance, virus stock aliquots were used to infect JC53-BL (US NIH Aids Reagent Program) cells yielding approximately 1.5×10⁵ RLU (relative light units) per well. Test peptide-immunoglobulin-conjugates, reference antibodies including the anti-CCR5 monoclonal antibody 2D7 (PharMingen; CCR5, chemokine receptor; coreceptor for HIV-1 infection) and reference peptides (T-651 and T-2635) were serially diluted in 96-well plates. The assay was carried out in quadruplicates. Each plate contained cell control and virus control wells. The equivalent of 1.5×10⁵ RLU of virus stocks were added to each well, then 2.5×10⁴ JC53-BL cells were added to each well, with a final assay volume of 200 μl per well. After three day incubation at 37° C., 90% relative humidity, and 5% CO₂, media were aspirated and 50 μl of Steady-Glo® Luciferase Assay System (Promega) was added to each well. The assay plates were read on a Luminometer (Luminoskan, Thermo Electron Corporation) after 10 minutes of incubation at room temperature. Percent inhibition of luciferase activity was calculated for each dose point after subtracting the background, and IC₅₀ was determined by using XLfit curve fitting software for Excel (version 3.0.5 Build12; Microsoft).

TABLE 7
Antiviral activity of peptides and peptide-immunoglobulin conjugates

| | Antiviral activity |
|---|---|
| | % Inhibition at 20 μg/mL |
| Compound | or IC₅₀ (μg/mL) |
| Reference antibody 1 (<IGF-1R>) | no activity |
| Reference antibody 2 (inert) | n.d. |
| Reference antibody 3 (inert) | n.d. |
| T-651 | 0.4 μg/mL |
| T-2635 | 0.5 μg/mL |
| Reference anti-CCR5 2D7 | 2.3 μg/mL |
| 4970/4802 | 20% |
| 4972/4802 | 43% |
| 4974/4802 | 12.5 μg/mL |
| 4976/4818 | 38% |
| 4965/4968 | 2.0 μg/mL |

### Example 8

### Single-Cycle Antiviral Assay

For the production of pseudotyped NL-Bal viruses, plasmid pNL4-3Δenv (HIV pNL4-3 genomic construct with a deletion within the env gene) and pCDNA3.1/NL-BAL env (pcDNA3.1 plasmid containing NL-Bal env gene (obtained from NIBSC Centralized Facility for AIDS Reagents)) were co-transfected into the HEK 293FT cell line (Invitrogen), cultured in Dulbecco's modified minimum medium (DMEM) containing 10% fetal calf serum (FCS), 100 U/mL Penicillin, 100 μg/mL Streptomycin, 2 mM L-glutamine and 0.5 mg/mL geniticin (all media from Invitrogen/Gibco). The supernatants containing pseudotyped viruses were harvested two days following transfection, and cellular debris was removed by filtration through a 0.45 μm pore size PES (polyethersulfone) filter (Nalgene) and stored at −80° C. in aliquots. For normalization in assay performance, virus stock aliquots were used to infect JC53-BL (US NIH Aids Reagent Program) cells yielding approximately 1.5×10⁵ RLU (relative light units) per well. Test peptide-immunoglobulin-conjugates, reference antibodies and reference peptides (T-20, T-651 and T-2635) were serially diluted in 96-well plates. The assay was carried out in quadruplicates. Each plate contained cell control and virus control wells. The equivalent of 1.5×10⁵ RLU of virus stocks were added to each well, then 2.5×10⁴ JC53-BL cells were added to each well, with a final assay volume of 200 μl per well. After 3 day incubation at 37° C., 90% Relative Humidity, and 5% CO₂, media were aspirated and 50 μl of Steady-Glo® Luciferase Assay System (Promega) was added to each well. The assay plates were read on a Luminometer (Luminoskan, Thermo Electron Corporation) after 10 minutes of incubation at room temperature. Percent inhibition of luciferase activity was calculated for each dose point after subtracting the background, and IC₅₀-values were determined by using XLfit curve fitting software for Excel (version 3.0.5 Build12; Microsoft).

TABLE 8
Antiviral activity of peptides and peptide-immunoglobulin conjugates

| | Antiviral activity |
|---|---|
| | % Inhibition at 20 μg/mL |
| Compound | or IC₅₀ (μg/mL) |
| Reference antibody 1 (anti-IGF- | no activity |
| 1R antibody) | |
| Reference antibody 2 (inert) | no activity |
| Reference antibody 3 (inert) | no activity |
| T-20 | 0.38 μg/mL |
| T-651 | 0.05 μg/mL |
| T-2635 | 0.05 μg/mL |
| Reference anti-CCR5 2D7 | 1.2 μg/mL |
| 4970/4802 | 15% |
| 4972/4802 | 27% |
| 4974/4802 | 18 μg/mL |
| 4976/4818 | 17% |
| 4965/4968 | 9 μg/mL |

### Example 9

### Antiviral Assay in Peripheral Blood Mononuclear Cells (PBMC)

Human PBMC were isolated from buffy-coats (obtained from the Stanford Blood Center) by a Ficoll-Paque (Amersham, Piscataway, N.J., USA) density gradient centrifugation according to manufacturer's protocol. Briefly, blood was transferred from the buffy coats in 50 ml conical tubes and diluted with sterile Dulbecco's phosphate buffered saline (Invitrogen/Gibco) to a final volume of 50 ml. Twenty-five ml of the diluted blood was transferred to two 50 ml conical tubes, carefully underlayerd with 12.5 ml of Ficoll-Paque Plus (Amersham Biosciences) and centrifuged at room temperature for 20 min. at 450×g without braking. The white cell layer was carefully transferred to a new 50 ml conical tube and washed twice with PBS. To remove remaining red blood cells, cells were incubated for 5 min. at room temperature with ACK lysis buffer (Biosource) and washed one more time with PBS. PBMC were counted and incubated at a concentration of 2-4×10⁶ cells/ml in RPMI1640 containing 10% FCS (Invitrogen/Gibco), 1% penicillin/streptomycin, 2 mM L-glutamine, 1 mM sodium-pyruvate, and 2 μg/ml Phytohemagglutinin (Invitrogen) for 24 h at 37° C. Cells were incubated with 5 Units/ml human IL-2 (Roche Molecular Biochemicals) for a minimum of 48 h prior to the assay. In a 96 well round bottom plate, 1×10⁵ PBMC were infected with the HIV-1 JR-CSF virus (Koyanagi, Y., et al. Science 236 (1987) 819-22) in the presence of serially diluted test peptide-immunoglobulin-conjugates, reference immunoglobulins and reference peptides (T-20, T-651 and T-2635). The amount of virus used was equivalent to 1.2 ng HIV-1 p24 antigen/well. Infections were set up in quadruplicates. Plates were incubated for 6 days at 37° C. Virus production was measured at the end of infection by using p24 ELISA (HIV-1 p24 ELISA #NEK050B) using the sigmoid dose-response model with one binding site in Microsoft Excel Fit (version 3.0.5 Build12; equation 205; Microsoft).

TABLE 9
Antiviral activity of peptides and peptide-immunoglobulin conjugates

| | Antiviral activity |
|---|---|
| Compound | IC₅₀ (μg/mL) |
| Reference antibody 1 (<IGF-1R>) | 6.7 |
| Reference antibody 2 (inert) | no activity |
| Reference antibody 3 (inert) | no activity |
| T-651 | 0.007 |
| T-2635 | 0.004 |
| 4965/4968 | 3.6 |
| 4974/4802 | 0.2 |

inert: antibody has a specific binding activity (K_{D} smaller than 10⁻⁸ mol/l) for an antigen that is not present in the assay

### Example 10

### Determination of the Binding Affinity of Polypeptides

Binding affinities of polypeptides based on the HR1-HR2 interaction of the HIV-1 gp41 protein (HR, Heptad Repeat 1 and 2 region) were measured by Surface Plasmon Resonance (SPR) using a BIAcore® 3000 instrument (Pharmacia, Uppsala, Sweden) at 25° C.

The BIAcore® system is well established for the study of molecule interactions. It allows a continuous real-time monitoring of ligand/analyte bindings and thus the determination of association rate constants (kₐ), dissociation rate constants (k_{d}), and equilibrium constants (K_{D}). SPR-technology is based on the measurement of the refractive index close to the surface of a gold coated biosensor chip. Changes in the refractive index indicate mass changes on the surface caused by the interaction of immobilized ligand with analyte injected in solution. If molecules bind immobilized ligand on the surface the mass increases, in case of dissociation the mass decreases.

Binding Assay

The Sensor Chip SA (SA, Streptavidin) was pre-washed by three consecutive 1-minute injections of 1 M NaCl in 50 mM NaOH. Then the biotinylated HR1 peptide Biotin-T-2324 (SEQ ID NO: 37) was immobilized on a SA-coated sensor chip. To avoid mass transfer limitations the lowest possible value (ca. 200 RU, Resonance Units) of HR1 peptide dissolved in HBS-P buffer (10 mM HEPES, pH 7.4, 150 mM NaCl, 0.005% (v/v) Surfactant P20) was loaded onto the SA-chip. Before the measurements were started the chip was regenerated a first time with a one minute pulse of 0.5% (w/v) sodium dodecyl sulfate (SDS) at a flow rate of 50 μL/min.

HR2 HIV-1 gp41 containing polypeptides to be analyzed were first dissolved in 50 mM NaHCO₃, pH 9, at a concentration of about 1 mg/mL and then diluted in HPS-P buffer to various concentrations ranging from 25 to 1.95 nM. The sample contact time was 5 min. (association phase). Thereafter the chip surface was washed with HBS-P for 5 min. (dissociation phase). All interactions were performed at exactly 25° C. (standard temperature). During a measurement cycle the samples were stored at 12° C. Signals were detected at a detection rate of one signal per second. Samples were injected at increasing concentrations at a flow rate of 50 μL/min over the HR1 coupled biosensor element. The surface was regenerated by 1 min washing with 0.5% (w/v) SDS solution at a flow rate of 50 μL/min.

The equilibrium constants (K_{D}), defined as kₐ/k_{d} were determined by analyzing the sensogram curves obtained with several different concentrations, using BIAevaluation 4.1 software package. Non specific binding was corrected by subtracting the response value of a HR2 containing polypeptide interaction with the free Streptavidin surface from the value of the HR2-HR1 interaction. The fitting of the data followed the 1:1 Langmuir binding model.

Deglycosylation of Polypeptides

HR2 containing polypeptide samples dissolved in PBS buffer at a concentration of about 2 mg/ml were deglycosylated (removal of N-glycans) with Peptide-N-Glycosidase F (PNGaseF, Prozyme, San Leandro, Calif.) by incubation at 37° C. for 12-24 h using 50 mU PNGaseF per mg N-glycosylated polypeptide.

TABLE 10a
Exemplary binding constants of HR2 containing polypeptides to HR1

| Sample | kₐ (1/Ms) | k_{d} (1/s) | K_{A} (1/M) | K_{D} (M) | Note/pharmacophor |
|---|---|---|---|---|---|
| T-20 | — | — | — | — | |
| T-1249 | 1.24 × 10⁶ | 1.27 × 10⁻³ | 9.80 × 10⁸ | 1.02 × 10⁻⁹ | |
| 4972 deg | 3.89 × 10⁵ | 6.59 × 10⁻⁵ | 5.89 × 10⁹ | 1.70 × 10⁻¹⁰ | T-2635 |
| 4974 deg | 4.01 × 10⁵ | 4.43 × 10⁻⁵ | 9.07 × 10⁹ | 1.10 × 10⁻¹⁰ | T-2635 |
| 4972 | 7.86 × 10⁵ | 7.76 × 10⁻⁵ | 1.01 × 10¹⁰ | 9.87 × 10⁻¹¹ | T-2635 |
| T-651 | 1.65 × 10⁶ | 1.41 × 10⁻⁴ | 1.17 × 10¹⁰ | 8.55 × 10⁻¹¹ | |
| T-2635 | 1.11 × 10⁶ | 7.38 × 10⁻⁵ | 1.50 × 10¹⁰ | 6.65 × 10⁻¹¹ | |
| 4970 deg | 5.28 × 10⁵ | 3.30 × 10⁻⁵ | 1.60 × 10¹⁰ | 6.26 × 10⁻¹¹ | T-651 |
| 4976 | 9.23 × 10⁵ | 3.51 × 10⁻⁵ | 2.63 × 10¹⁰ | 3.80 × 10⁻¹¹ | T-651 |
| 4965/4968 | 2.69 × 10⁵ | 7.91 × 10⁻⁶ | 3.41 × 10¹⁰ | 2.93 × 10⁻¹¹ | 65(T-651)/68(T-2635) |
| deg | | | | | |
| 4976 deg | 5.12 × 10⁵ | 9.75 × 10⁻⁶ | 5.25 × 10¹⁰ | 1.90 × 10⁻¹¹ | T-651 |
| 4970 | 1.01 × 10⁶ | 1.11 × 10⁻⁵ | 9.11 × 10¹⁰ | 1.10 × 10⁻¹¹ | T-651 |
| 4974 | 4.37 × 10⁵ | 4.75 × 10⁻⁷ | 9.20 × 10¹¹ | >1 × 10⁻¹² | T-2635 |
| 4965 deg | 4.76 × 10⁵ | 3.24 × 10⁻⁷ | 1.47 × 10¹² | >1 × 10⁻¹² | T-651 |
| 4965/4968 | 3.69 × 10⁵ | 8.49 × 10⁻⁸ | 4.35 × 10¹² | >1 × 10⁻¹² | 65(T-651)/68(T-2635) |
| 4965 | 4.61 × 10⁵ | 6.49 × 10⁻⁸ | 7.11 × 10¹² | >1 × 10⁻¹² | T-651 |

deg: deglycosylated with PNGaseF

[image]

[image]

[image]

What is claimed:

1. A peptide-immunoglobulin-conjugate, comprising:
a first immunoglobulin heavy chain;
a second immunoglobulin heavy chain;
a first biologically active peptide; and
a second biologically active peptide;
wherein said first immunoglobulin heavy chain and said second immunoglobulin heavy chain are the same or different, and said first peptide and said second peptide are the same or different;
wherein said first and second polypeptides are biologically active, and are fused at either the N-terminal or C-terminal of said first and second immunoglobulin heavy chains;
and wherein said first and second immunoglobulin heavy chains are non-functional, and form a pair.

2. The peptide-immunoglobulin-conjugate of claim 1, further comprising:
a first immunoglobulin light chain polypeptide; and
a second immunoglobulin light chain polypeptide;
wherein said first and second immunoglobulin light chains are non-functional.

3. The peptide-immunoglobulin-conjugate of claim 2, wherein at least one of said first immunoglobulin light chain polypeptide and said second immunoglobulin light chain polypeptide further comprises a third biologically active peptide fused to either the N-terminal or C-terminal of said immunoglobulin light chain.

4. A peptide-immunoglobulin-conjugate, comprising:
a first immunoglobulin heavy chain;
a second immunoglobulin heavy chain;
a first immunoglobulin light chain;
a second immunoglobulin light chain;
a first biologically active peptide; and
a second biologically active peptide;
wherein said first immunoglobulin heavy chain and said second immunoglobulin heavy chain are the same or different, said first immunoglobulin light chain and said second immunoglobulin light chain are the same or different and said first peptide and said second peptide are the same or different;
wherein said first and second polypeptides are biologically active, and are fused at either the N-terminal or C-terminal of one or more immunoglobulin light or heavy chains;
and wherein said first and second immunoglobulin heavy chains are non-functional, and form a pair, and said first and second immunoglobulin light chains are non-functional, and bind to said first and second immunoglobulin heavy chains.

5. The peptide-immunoglobulin-conjugate of claim 1, wherein one of said first peptide and said second peptide further comprises a peptidic linker, wherein said peptidic linker joins said first or second peptide with said first or second immunoglobulin heavy chain.

6. The peptide-immunoglobulin-conjugate of claim 4, wherein one of said first peptide and said second peptide further comprises a peptidic linker, wherein said peptidic linker joins said first or second peptide with said first or second immunoglobulin heavy chain or first or second immunoglobulin light chain.

7. The peptide-immunoglobulin-conjugate of claim 1, wherein said first and second peptide are at least 90% identical in sequence.

8. The peptide-immunoglobulin-conjugate of claim 3, wherein all said biologically active peptides are at least 90% identical in sequence.

9. The peptide-immunoglobulin-conjugate of claim 4, wherein said first and second peptide are at least 90% identical in sequence.

10. The peptide-immunoglobulin-conjugate of claim 1, wherein said first and second immunoglobulin heavy chains are IgG or IgE.

11. The peptide-immunoglobulin-conjugate of claim 4, wherein said first and second immunoglobulin heavy chains are IgG or IgE.

12. The peptide-immunoglobulin-conjugate of claim 1, wherein at least one of said biologically active peptides is an antifusogenic peptide.

13. The peptide-immunoglobulin-conjugate of claim 4, wherein at least one of said biologically active peptides is an antifusogenic peptide.

14. The peptide-immunoglobulin-conjugate of claim 1, wherein said first and second immunoglobulin heavy chains bind a human antigen with a K_{D}-value (binding affinity) of 10⁻⁵ mol/l or higher.

15. The peptide-immunoglobulin-conjugate of claim 1, wherein said first and second immunoglobulin heavy chains lack a part or all of one or more framework or/and hypervariable regions.

16. The peptide-immunoglobulin-conjugate of claim 1, wherein said first and second immunoglobulin heavy chains lack a part of the variable regions.

17. The peptide-immunoglobulin-conjugate of claim 2, wherein said first and second immunoglobulin heavy chains and said first and second immunoglobulin light chains lack a part or all of one or more framework or/and hypervariable regions.

18. The peptide-immunoglobulin-conjugate of claim 2, wherein said first and second immunoglobulin heavy chains and said first and second immunoglobulin light chains lack a part of the variable regions.

19. The peptide-immunoglobulin-conjugate of claim 4, wherein said first and second immunoglobulin heavy chains bind a human antigen with a K_{D}-value (binding affinity) of 10⁻⁵ mol/l or higher, and said first and second immunoglobulin light chains bind a human antigen with a K_{D}-value (binding affinity) of 10⁻⁵ mol/l or higher.

20. The peptide-immunoglobulin-conjugate of claim 4, wherein said first and second immunoglobulin heavy chains and said first and second immunoglobulin light chains each lack a part or all of one or more framework or/and hypervariable regions.

21. The peptide-immunoglobulin-conjugate of claim 4, wherein said first and second immunoglobulin heavy chains and said first and second immunoglobulin light chains each lack a part the variable regions.

22. A method for producing a conjugate according to claim 1, said method comprising:
a) providing a host cell comprising one or more nucleic acid molecules encoding a peptide immunoglobulin-conjugate according to claim 1;
b) culturing said host cell under conditions suitable for the expression of the peptide immunoglobulin-conjugate; and
c) recovering the peptide immunoglobulin-conjugate from the cell or culture medium.

23. A method for producing a conjugate according to claim 4, said method comprising:
a) providing a host cell comprising one or more nucleic acid molecules encoding a peptide-immunoglobulin-conjugate according to claim 4;
b) culturing said host cell under conditions suitable for the expression of the peptide-immunoglobulin-conjugate; and
c) recovering the peptide immunoglobulin-conjugate from the cell or culture medium.

24. A pharmaceutical composition, comprising:
an effective amount of a peptide-immunoglobulin-conjugate according to claim 1; and
a pharmaceutically acceptable excipient.

25. A pharmaceutical composition, comprising:
an effective amount of a peptide-immunoglobulin-conjugate according to claim 4; and
a pharmaceutically acceptable excipient.

26. A method of treating a viral infection in a patient, said method comprising:
administering an effective amount of a peptide-immunoglobulin-conjugate according to claim 1 to a patient in need thereof.

27. The method of claim 26, wherein said first or second peptide competes with the virus causing said viral infection for binding to a cellular receptor.

28. The method of claim 26, wherein said viral infection is HIV infection.

29. The method of claim 28, wherein said first or second peptide comprises an antifusogenic peptide.

30. A method of treating a viral infection in a patient, said method comprising:
administering an effective amount of a peptide-immunoglobulin-conjugate according to claim 4 to a patient in need thereof.

31. The method of claim 30, wherein at least one of said biologically active peptides competes with the virus causing said viral infection for binding to a cellular receptor.

32. The method of claim 30, wherein said viral infection is HIV infection.

33. The method of claim 32, wherein said biologically active peptide comprises an antifusogenic peptide.
